(19) United States
(12) Patent Application Publication
(10) Pub. No.: US 2025/0109120 A1
(43) Pub. Date: Apr. 3, 2025

(54) INDOLINE DERIVATIVES FOR TREATMENT AND/OR PREVENTION OF TUMOR OR CELL PROLIFERATIVE AND FIBROSIS DISEASES

(71) Applicants: NATIONAL TAIWAN UNIVERSITY, Taipei (TW); Taipei Medical University, Taipei City (TW)

(72) Inventors: Chia-Ron YANG, Taipei (TW); Wei-Jan HUANG, Taipei City (TW); Kai-Cheng HSU, New Taipei City (TW)

(21) Appl. No.: 18/477,022

(22) Filed: Sep. 28, 2023

(51) Int. Cl. C07D 401/12; A61P 11/00; A61P 35/04

(52) U.S. Cl. CPC C07D 401/12; A61P 11/00; A61P 35/04

(57) ABSTRACT

The invention relates to indoline derivatives and uses thereof for treating and/or preventing an inflammatory condition or fibrosis diseases, and tumor or cell proliferative diseases.

### FIELD OF THE INVENTION

The present disclosure relates to indoline derivatives and uses thereof for treating and/or preventing an inflammatory condition or fibrosis diseases, and tumor or cell proliferative diseases.

### BACKGROUND OF THE INVENTION

Cyclin-dependent protein kinase 8 (CDK8) is a nuclear serine-threonine protein kinase that regulates several transcription factors and pathways, such as Wnt/β-catenin, TGF-β/Smad, and STAT. Among them, TGF-β and the involvement thereof in inflammatory processes are considered essential for initiating pulmonary fibrosis. Therefore, CDK8 not only plays important roles in regulating fibrotic growth factors and inflammatory signaling pathways, but also in transcriptional regulation.

There has been a recent increase in the investigation of small molecule inhibitors targeting CDK8, and many of the studies have indicated the promising role of CDK8 as a target in the treatment of various cancers such a as colorectal cancers, metastatic prostate cancer, acute myeloid leukemia and advanced ER(+) HER2(−) metastatic breast cancer, as well as anti-inflammatory and fibrotic therapy.

However, it has been observed that these CDK8 inhibitors exhibit metabolic instability and unspecific cytotoxicity, which potentially leads to adverse events in treated patients, highlighting an urgent need to find new therapeutic targets to improve existing treatments and unmet medical needs.

### SUMMARY OF THE INVENTION

The present disclosure provides a compound of Formula (I):

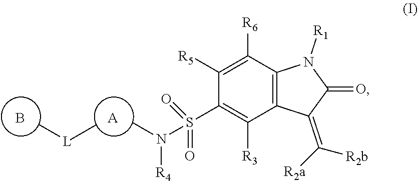

or a pharmaceutically acceptable salt, stereoisomers, enantiomers, prodrugs, hydrates or solvates thereof, wherein:
R¹ is hydrogen, C₁₋₆ alkyl, C₁₋₆ haloalkyl, C₁₋₆ alkoxy, C₁₋₆ haloalkoxy or phenyl;
R^{2a} and R^{2b} each independently are C₁₋₆ alkyl, C₁₋₆ alkenyl or C₁₋₆ alkynyl, which is optionally substituted with halogen, or
R^{2a} and R^{2b} taken together with the carbon atom to which they attach form Ring C, wherein Ring C is 4-6 membered cycloalkyl or 4-6 membered heterocycloalkyl, wherein the 4-6 membered cycloalkyl or 4-6 membered heterocycloalkyl is optionally substituted with phenyl or benzyl, in which said phenyl or benzyl is optionally substituted with halogen or hydroxy;
each of R³, R⁵, and R⁶, independently is, H, C₁₋₆alkyl, C₂₋₄alkenyl, C₂₋₆alkynyl, 6-10 membered aryl, 5-10 membered heteroaryl, 3-8 membered cycloalkyl, 3-8 membered cycloalkenyl, 3-8 membered heterocycloalkyl, 3-8 membered heterocycloalkenyl, halo, cyano, nitro, ORₐ, SRₐ, S(O)Rₐ, SO₂Rₐ, CH═CH—C(O)NR_{b}R_{c}, NHC(O)—CH═CH—C(O)Rₐ, NHC(O)—CH═CH—C(O)NR_{b}R_{c}, SO₂NR_{b}R_{c}, OC(O)R, C(O)NR_{b}R_{c}, NR_{b}R_{c}, NHC(O)Rₐ, NHC(O)NR_{b}R_{c}, or NHC(S)R_{b}, wherein each of Rₐ, R_{b}, and R_{c}, independently, is H, hydroxy, C₁₋₆alkoxy, 6-10 membered aryloxy, 5-10 membered heteroaryloxy, C₁₋₆alkyl, C₂₋₆alkenyl, C₂₋₆alkynyl, 6-10 membered aryl, 5-10 membered heteroaryl, 3-8 membered cycloalkyl, 3-8 membered cycloalkenyl, 3-8 membered heterocycloalkyl, or 3-8 membered heterocycloalkenyl; and
R⁴ is hydrogen, C₁₋₁₀ aliphatic group, phenyl or 5-6 membered heteroaryl, which is optionally substituted with one or more substituents selected from the group consisting of halogen, hydroxy, nitro, cyano, amino, phenyl, 5-6 membered heteroaryl, C₁₋₆ alkoxy and C₁₋₆ haloalkoxy;
Ring A is phenyl, a diazole, a thiadiazole, a thiazolobenzene or a benzopiperazine, which is optionally substituted with one or more substituents selected from the group consisting of oxo, halogen, C₁₋₆alkyl, phenyl, a diazole and C₁₋₃alkylaminocarbonyl, wherein the substituent C₁₋₆alkyl, phenyl and diazole is optionally further substituted with halogen, C₁₋₃alkyl or any combinations thereof;
-L- is —O—, —NR^{a}—, —C(═O)—, —NR^{a}—C(═O)— or —C(═O)—NR^{a}—, wherein R^{a} has the meaning as defined above; and
Ring B is phenyl, a diazole or pyridine, which is optionally substituted with one or more substituents selected from the group consisting of nitro, halogen, alkyl, pyridine and C₁₋₃alkylaminocarbonyl; or
-L-B is absent.

In various embodiments, in the compound of Formula (I), R¹ is hydrogen and R^{2a} and R^{2b} each independently are methyl. In various embodiments, Ring A is substituted with halogen.

In one embodiment, in the compound of Formula (I), Ring A is phenyl, a diazole, a thiadiazole, a thiazolobenzene or a benzopiperazine, which is optionally substituted with one or more substituents selected from the group consisting of oxo, halogen, C₁₋₆alkyl, phenyl, a diazole and C₁₋₃alkylaminocarbonyl, wherein the substituent C₁₋₆alkyl, phenyl and diazole is optionally further substituted with halogen, C₁₋₃alkyl or any combinations thereof;
-L- is —O—, —NR^{a}— or —C(═O)—; and
Ring B is phenyl, a diazole or pyridine, which is optionally substituted with one or more substituents selected from the group consisting of halogen, alkyl and C₁₋₃alkylaminocarbonyl; or
-L-B is absent.

In one embodiment, in the compound of Formula (I), Ring A is phenyl optionally substituted with one or more substituents selected from the group consisting of halogen and C₁₋₆alkyl; L is —O—, —NH—, —C(═O)— or —C(═O)—NR^{a}— wherein Ring A is linked to the N atom; and Ring B is phenyl or pyridine which is optionally substituted with one or more substituents selected from the group consisting of nitro, halogen, C₁₋₆alkyl and C₁₋₃alkylaminocarbonyl.

In one embodiment, in the compound of Formula (I), Ring A is phenyl, a diazole or a thiadiazole, which is substituted with one or more substituents selected from the group consisting of phenyl, a diazole or C₁₋₃alkylaminocarbonyl, and the substituent phenyl and diazole is optionally further substituted with C₁₋₃alkyl; and -L-B is absent.

In one embodiment, in the compound of Formula (I), Ring A is a thiazolobenzene or a benzopiperazine, which is optionally substituted with one or more substituents selected from the group consisting of oxo, halogen and alkyl; and -L-B is absent.

In one embodiment, in the compound of Formula (I), Ring A is phenyl, which is optionally substituted with one or more substituents selected from the group consisting of halogen and alkyl; -L- is —NH—; and Ring B is pyridine which is optionally substituted with one or more substituents selected from the group consisting of halogen and C₁₋₆alkyl.

In one embodiment, in the compound of Formula (I), Ring A is phenyl substituted with halogen; -L- is —O—; Ring B is pyridine substituted with C₁₋₃alkylaminocarbonyl.

In one embodiment, the compound of Formula (I) has formula (II):

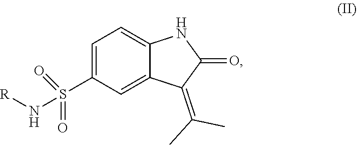

wherein R is selected from the group consisting of:

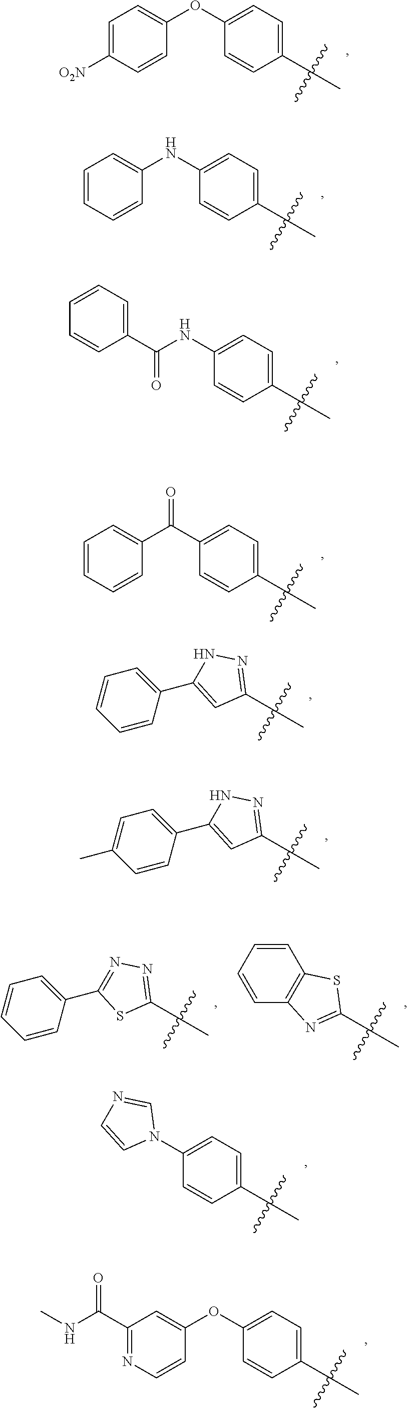

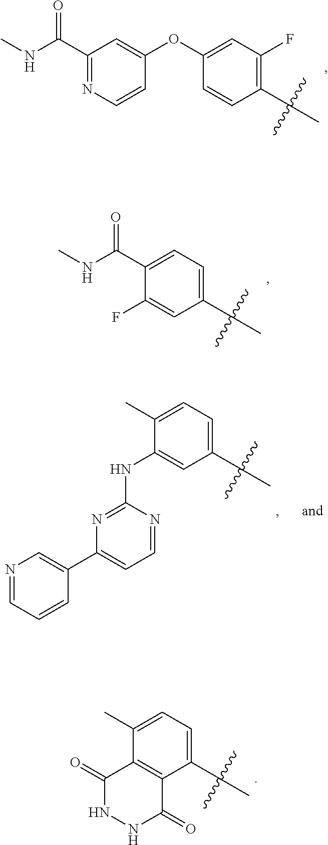 [SMILES: *c1ccc(Oc2ccnc(C(=O)NC)c2)cc1F ; *c1ccc(C(=O)NC)c(F)c1 ; *c1ccc(C)c(Nc2nccc(-c3cccnc3)n2)c1 ; *c1ccc(C)c2c(=O)[nH][nH]c(=O)c12]

In one embodiment, in the compound of Formula (I), Ring C is cyclobutane, cyclohexane or piperidine, and Ring C is optionally substituted with benzyl or phenyl, wherein said benzyl or phenyl is optionally substituted with hydroxy.

In one embodiment, the compound of Formula (I) has formula (III):

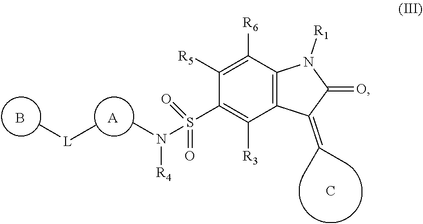

wherein R¹, R³, R⁴, R⁵, R⁶, Ring A, Ring B and L are as defined above, and Ring C is selected from the group consisting of:

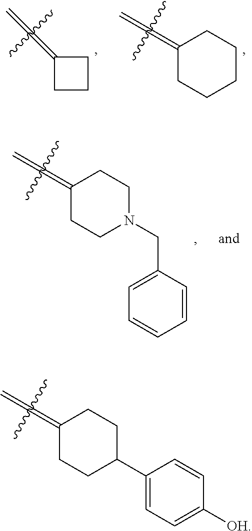 [SMILES: *=C1CCC1 ; *=C1CCCCC1 ; *=C1CCN(Cc2ccccc2)CC1 ; *=C1CCC(c2ccc(O)cc2)CC1]

The present disclosure also provides a pharmaceutical composition which comprises a compound or a pharmaceutically acceptable salt, stereoisomers, enantiomers, prodrugs, hydrates and solvates thereof, and a pharmaceutically acceptable carrier.

The present disclosure also provides a method of inhibiting CDK8 in a subject, which comprises administering a therapeutically effective amount of the compound or a pharmaceutically acceptable salt, stereoisomers, enantiomers, prodrugs, hydrates and solvates thereof to the subject.

The present disclosure also provides a method of preventing or treating an inflammatory condition and/or fibrosis diseases, which comprises administering a therapeutically effective amount of the compound as disclosed herein or a pharmaceutically acceptable salt, stereoisomers, enantiomers, prodrugs, hydrates and solvates thereof to a subject in need thereof.

The present disclosure also provides a method of preventing or treating tumor and/or cell proliferative disease, comprising administering a therapeutically effective amount of the compound as disclosed herein or a pharmaceutically acceptable salt, stereoisomers, enantiomers, prodrugs, hydrates and solvates thereof to a subject in need thereof.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1 shows the 2D interaction pose of compound k. 2D interaction pose of compound k. Red dashed lines denote hydrogen bonds. Hydrophobic interactions are represented as green lines. The 2D interactions were created in LeadIT.

FIGS. 2A-2D show the evaluation of CDK8 inhibition by compound k. FIG. 2A shows the inhibition efficiency of compound k against a panel of 54 kinases from different kinase families. FIG. 2B shows that DU145 and PC-3 cells were incubated with or without different concentrations (0.1, 0.3, 1, 3, 10, 30 μM) of compound k for 24 and 72 h. Cell viability was measured by MTT assay, and the 50% inhibitory concentration (IC50) values were calculated by a sigmoidal dose-response equation. FIGS. 2C and 2D show that DU145 and PC-3 cells were transfected with or without the pcDNA3 CDK8-HA plasmid (1 gg) for 24 h and then treated with the indicated concentrations of compound k for another 24 h. Whole-cell lysates were subjected to western blotting with the indicated antibodies. The results are shown as the mean t SEM from at least three independent experiments (*p<0.05, **p<0.01, and ***p<0.001 vs. control group; #p<0.05 vs. relevant control).

FIGS. 3A-3H show that CDK8 expression and TGF-β signals are increased in metastatic prostate cancer and are associated with poor patient survival. FIG. 3A shows the box plots comparing CDK8 mRNA expression in primary and metastatic prostate cancer samples from the GSE74685 dataset. FIG. 3B shows the comparison of CDK8 mRNA transcript levels in lymph node-positive vs. lymph node-negative prostate cancer samples from The Cancer Genome Atlas (TCGA) database. FIG. 3C shows the Kaplan-Meier plots depicting the time until biochemical relapse in radical proctectomy samples with high vs. low CDK8 expression, based on data from the GSE70769 dataset. FIG. 3D shows the gene set enrichment analysis (GSEA) of mediator complex components in metastatic vs. primary prostate cancer samples from the GSE68882 dataset. FIGS. 3E and 3F show the GSEA of TGF-β/Smad signaling pathway genes in prostate cancer patients grouped according to a Gleason score cutoff of 7 (medium grade) was performed using patient samples provided by Abida et al. and the GSE16560 dataset. FIG. 3G shows the Kaplan-Meier plots illustrating 10-year progression-free survival in prostate cancer patients with high vs. low TGFB1 expression based on the TCGA dataset. FIG. 3H shows the Box plots comparing TGFBR1 mRNA expression in primary vs. metastatic prostate cancer patients using samples from the GSE74685 dataset.

FIGS. 4A-4B show the inhibition of epithelial-to-mesenchymal transition protein expressions by CDK8 inhibitors in human alveolar epithelial cells. FIG. 4A shows that A549 cells were treated test compounds (2 μM), senexin A (5 μM), or pirfenidone (1 mM) for 1 h and stimulated with TGF-β1 (10 ng/mL) for a further 1 h. Cell lysates were subjected to a Western blot analysis using the indicated antibodies. Results are shown as the mean t SEM from three independent experiments. *p<0.05, and ***p<0.001 compared to the control group; #p<0.05, ##p<0.01, and ###p<0.001 compared to the TGF-β1-treated group. FIG. 4B show that cells were incubated for 12, 24, or 48 h with or without the indicated concentrations of compound k. Cell viabilities were determined by an MTT assay. Results are shown as the mean t SEM of three independent experiments.

FIGS. 5A-5B show that compound k inhibited the migration of A549 cells. A549 cells were incubated in six-well plates until reaching 90% confluency, scratched with a pipette tip, and immediately photographed (0 h). Cells were incubated with or without compound k (2 μM), senexin A (5 μM), or pirfenidone (1 mM) for 1 h, then treated with TGF-β1 (10 ng/mL) and allowed to migrate into the wound area for 24 or 48 h, and photographed. Cell migration into the wound was quantified using ImageJ software; the solid line indicates time zero. Quantitative assessment of the mean number of cells in the denuded zone is expressed as the mean±SEM from three independent experiments. **p<0.01 compared to the control group; ##p<0.01 and ###p<0.001 compared to TGF-β1-treated group.

FIGS. 6A-6D show that cell migration and EMT features of prostate cancer cells were notably suppressed by compound k. FIG. 6A shows that DU145 cells were treated with or without compound k at the indicated concentrations for 24 h in the presence of TGF-β1 (10 ng/mL). Images of the morphology of cells were taken at ×100 magnification. FIG. 6B shows that DU145 cells were incubated in 6-well plates until reaching 90% confluency, scratched with a pipette tip, and immediately photographed (0 h). The cells were incubated with or without TGF-β1 (10 ng/mL) and compound k (2.5 and 10 μM), allowed to migrate into the wound area for 48 h and photographed. Cell migration into the wound was quantified using ImageJ software; the white solid line indicates time zero. The quantitative assessment of the mean number of cells in the scratch area is expressed as the mean t SEM from at least three independent experiments (***p<0.001 vs. control group; ##p<0.01 and ###p<0.001 vs. TGF-β1 treatment group). (C) PC-3 cells were incubated with 10 μM compound k/senexin A for 1 h, subsequently treated with TGF-β1 (10 ng/mL) for 24 h and incubated with the indicated antibodies and DAPI. The immunofluorescence images were photographed by a ZEISS ApoTome.2 microscope. Images were taken at 600× magnification.

FIGS. 7A-7F show that compound k significantly inhibited TGF-β1-induced EMT proteins and gene expression. Cells were exposed to compound k as indicated concentrations or senexin A (10 μM) in the presence or absence of TGF-β1 (10 ng/mL) for 24 h, the proteins and mRNA levels of E-cadherin, N-cadherin, Snail, vimentin, α-SMA were determined by western blot (A) or real-time PCR (B). Results are shown as the mean±SEM from at least three independent experiments (**p<0.01, ***p<0.001 vs. control group; #p<0.05, ##p<0.01, and ###p<0.001 vs. TGF-β1 treatment group).

FIGS. 8A-8B show the inhibitory effects of cellular distributions of TGF-β1-induced p-Smad3 T179, RNA polymerase II pS2/S5, and Smad3 after treatment with compound k. Human alveolar epithelial A549 cells were incubated with compound k (5 μM), senexin A (5 μM), or pirfenidone (1 mM) for 1 h, followed by stimulation with TGF-β1 (10 ng/ml) for 3 h. Protein expressions in the cytoplasmic and nuclear fractions were subjected to a Western blot analysis using the indicated antibodies. α-Tubulin and histone H3 served as loading controls for the cytoplasmic and nuclear fractions, respectively. Results are shown as the mean±SEM of three independent experiments. **p<0.01 and ***p<0.001 compared to the control group; #p<0.05 and ##p<0.01 compared to the TGF-β1-treated group.

FIGS. 9A-9G show that compound k significantly inhibited TGF-β/Smad/RNA polymerase II signaling. FIGS. 9A-9B show that human prostate cancer DU145 cells were transfected with the pcDNA3 CDK8-HA plasmid (1 μg) for 24 h, treated with compound k (10 μM) for 1 h and then incubated with TGF-β1 (10 ng/mL) for an additional 24 h. Whole-cell lysates were subjected to western blotting with the indicated antibodies. FIGS. 9C-9D show that DU145 cells were treated with TGF-β1 (10 ng/mL) with or without compound k (10 μM) for 1 h and then subjected to nuclear-cytosolic fractionation. The proteins in the cytosol and nucleus were detected by western blotting analysis, and the proteins expression in the nucleus were quantified. FIGS. 9E-9F show that DU145 cells were incubated with compound k (10 μM) for 1 h with or without TGF-β1 (10 ng/mL), and the total cell lysates were immunoprecipitated with antibodies and subjected to immunoblotting. FIG. 9G shows that DU145 cells were transfected with 7TFP CDH1 reporter plasmid (1 μg) for 24 h followed by subsequent treatment with TGF-β1 (10 ng/mL) with or without compound k and senexin A (10 μM) for 24 h, and luciferase expression was subsequently determined. The results are shown as the mean±SEM from at least three independent experiments (*p<0.05, **p<0.01, and ***p<0.001 vs. control group; #p<0.05, ##p<0.01, and ###p<0.001 vs. TGF-β1 treatment group; § p<0.05, §§ p<0.01 vs. indicated group).

FIGS. 10A-10B show that CDK8 inhibition reduced TGF-β1-induced oxidative stress. A549 cells were incubated with compounds k, b, a (2 μM), senexin A (5 μM), or pirfenidone (1 mM) for 1 h, followed by treatment with 10 ng/ml TGF-β1 for a further 24 h. Malondialdehyde (MDA) (FIG. 10A) and Nox4 (FIG. 10B) levels were detected. ***p<0.001 compared to the control group; #p<0.05, ##p<0.01, and ###p<0.001 compared to the TGF-β1-treated group.

FIGS. 11A-11L show that compound k hindered TGF-β1-induced β-catenin signaling. FIGS. 11A-11B show that DU145 cells were incubated with TGF-β1 (10 ng/ml) for 24 h, and whole-cell lysates were collected and for protein concentration assessment as indicated. FIGS. 11C-11D show that cells were incubated with compound k (10 μM) for 12 h and with or without TGF-β1 (10 ng/ml) for another 2 h, and the nuclear lysates were immunoprecipitated with antibodies and subjected to immunoblotting. FIG. 11E shows that gene expression comparison between CDK8 and β-catenin in prostate cancer patients by TCGA database analysis (n=498). FIGS. 11F-11G show that DU145 cells were incubated with 20 mM LiCl for 6 h. Inhibitory phosphorylation of GSK3β was evaluated through western blotting. FIGS. 11H-11I show that DU145 cells were treated with compound k at the indicated concentrations and senexin A (10 μM) for 20 h and then incubated with or without LiCl (20 mM) for another 6 h. Whole-cell lysates were subjected to western blotting with the indicated antibodies. FIGS. 11J-11K show the comparison of basal activities of the Akt/GSK3β axis between PC-3 and DU145 cells. FIG. 11L shows the concentration-dependent inhibition of compound k on β-catenin target proteins in PC-3 cells following 24 h of treatment with compound k. The results are shown as the mean±SEM from at least three independent experiments (*p<0.05, **p<0.01, ***p<0.001 vs. control group; #p<0.05, ##p<0.01, and ###p<0.001 vs. relevant control).

FIGS. 12A-12G show that compound k exhibited anti-invasion effects in vivo. PC-3 cells (1×107 cells) were injected subcutaneously into the flanks of 7-week-old male SCID mice and treated with vehicle or compound k (50 mg/kg) q.d. for 31 days. FIG. 12A shows that changes in body weight were then recorded during compound treatment (n=7, mean±SEM). Tumors were excised from each mouse after a 31-day treatment. FIGS. 12B-12C show that paraffin sections of tumors were stained with hematoxylin and eosin and antibodies as indicated. The length is represented by the scale bar as indicated. FIGS. 12D-12G show that immunoblots and immunoprecipitation assay of tumor samples were analyzed with antibodies as indicated.

### DETAILED DESCRIPTION OF THE PRESENT DISCLOSURE

The present disclosure is, at least in part, based on the discovery of using an indoline derivative and its effective dose in the prevention and/or treatment of an inflammatory condition or fibrosis diseases. The compound can effectively prevent and/or treat an inflammatory condition or fibrosis disease without cytotoxicity or genotoxicity.

As used herein, except where the context requires otherwise, the term “comprise” and variations of the term, such as “comprising,” “comprises” and “comprised” are not intended to exclude other additives, components, integers or steps.

As used herein, except where the context requires otherwise, the method steps disclosed are not intended to be limiting nor are they intended to indicate that each step is essential to the method or that each step must occur in the order disclosed.

As used herein, the use of “or” means “and/or” unless stated otherwise. In the context of a multiple dependent claim, the use of “or” refers back to more than one preceding independent or dependent claim in the alternative only.

As used herein, all numbers are approximate, and may be varied to account for measurement error and the rounding of significant digits. The use of “about” before certain measured quantities includes variations due to sample impurities, measurement error, human error, and statistical variation, as well as the rounding of significant digits.

As used herein, the term “about” refers to an acceptable deviation of a given value measured by a person of ordinary skill in the art, depending, in part, on how to measure or determine the value.

As used herein, the term “aliphatic” or “aliphatic group” may include saturated or unsaturated, linear, branched or cyclic groups, e.g., (cyclo) alkanes, (cyclo) alkenes, (cyclo) alkynes, etc. In certain cases, the aliphatic groups may contain heteroatom(s) interrupted in the chain or acting as a ring member atom.

As used herein, the term “alkyl” refers to a saturated, straight or branched alkyl, which comprises preferably 1-20 carbon atoms, and more preferably 1-6 carbon atoms, or the number of carbon atoms is specified. Examples of alkyl include, but are not limited to, methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, octadecyl or the like.

As used herein, the term “alkoxyl” or “alkoxy” as used herein means a group having a formula “—O-alkyl,” wherein the definition of the “alkyl” in said formula has the meaning of “alkyl” as stated above.

As used herein, the term “cycloalkyl” as used herein means a saturated or partially unsaturated cyclic carbon radical containing 3 to 10 ring carbon atoms and more preferably 3 to 8 ring carbon atoms, and optionally an alkyl substituent(s) on the ring. Examples of cycloalkyl include, but are not limited to, cyclopropyl, cyclopropenyl, cyclobutyl, cyclopentyl, cyclohexyl, 2-cyclohexen-1-yl, and the like.

As used herein, the term “aromatic,” “aromatic group” or “aryl” refers to those having aromaticity, which may have 6 to 12 ring members and may have a mono cyclic ring or a polycyclic (fused) rings. Examples of the aromatic groups include, but are not limited to, phenyl, benzyl, biphenyl, naphthalene, fluorene, etc.

As used herein, the term “heteroaryl” refers to those having aromaticity, which may have 5 to 12 ring members and at least one heteroatom, preferably selected from nitrogen, oxygen and sulfur, as the ring member and may have a mono cyclic ring or a polycyclic (fused) rings. Examples of the aromatic groups include, but are not limited to pyrrole, pyridine, thiene, oxazole, furan, pyrimidine, diazoles, triazoles, thiadiazoles, thiazolobenzenes, benzopiperazine, etc.

As used herein, the term “halogen” or “halo” denotes fluorine, chlorine, bromine or iodine.

As used herein, the term “amino” as used herein means a functional group of the formula-NR′R″, wherein R′ and R″ each independently represent hydrogen, an aliphatic group or an aromatic group as defined above.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal and ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical/chemical association may include hydrogen bonding. Conventional solvents include water (i.e., forming a hydrate), methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “prodrug” refers to compounds, including derivatives of the compounds described herein, which have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offer advantages of solubility, tissue compatibility, or delayed release in the mammalian organism. Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides.

The terms “administer,” “administering,” or “administration,” as used herein, refers to implanting, absorbing, ingesting, injecting, inhaling, or otherwise introducing an inventive compound, or a pharmaceutical composition thereof, in or on a subject.

As used herein, the terms “condition,” “disease,” and “disorder” are used interchangeably.

An “effective amount” of a compound described herein refers to an amount sufficient to elicit the desired biological response, i.e., treating the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent.

As used herein, the term “pharmaceutically acceptable carrier” refers to a solid, semisolid, or liquid filler, diluent, encapsulating material, formulation auxiliary, or carrier conventional in the art for use with a therapeutic agent for administration to a subject. A pharmaceutically acceptable carrier is non-toxic to recipients at the dosages and concentration employed, and is compatible with other ingredients of the formulation. Pharmaceutically acceptable carriers are determined in part by the particular composition being administered, as well as by the particular method used to administer the composition.

As used herein, the term “subject” is defined herein to include animals such as mammals, including, but not limited to, primates (e.g., humans), cows, sheep, goats, horses, dogs, cats, rabbits, rats, mice and the like. In specific embodiments, the subject is a human. The terms “subject” and “patient” are used interchangeably herein in reference, for example, to a mammalian subject, such as a human.

As used herein, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound or dosage form provided herein, with or without one or more additional active agents, after the diagnosis or onset of symptoms of the particular disease.

As used herein, the terms “prevent,” “preventing” and “prevention” refer to the prevention of the onset, recurrence or spread of a disease or disorder, or of one or more symptoms thereof. In certain embodiments, the terms refer to the treatment with or administration of a compound or an antibody or dosage form provided herein, with or without one or more other additional active agents, prior to the onset of symptoms, particularly to patients at risk of disease or disorders provided herein. The terms encompass the inhibition or reduction of a symptom of the particular disease.

As used herein, the terms “co-administration” and “in combination with” include the administration of two or more therapeutic agents simultaneously, concurrently, separately or sequentially within no specific time limits unless otherwise indicated. In one embodiment, the therapeutic agents are in the same composition or unit dosage form. In other embodiments, the therapeutic agents are in separate compositions or unit dosage forms.

The inventors found that specific indoline derivative compounds may exhibit superior effects in inhibiting CDK8 and potential in treating CDK8-associated diseases. Hence, in one aspect, the present disclosure is directed to a compound of formula (I):

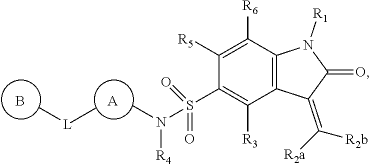

or a pharmaceutically acceptable salt, stereoisomers, enantiomers, prodrugs, hydrates or solvates thereof, wherein the definition of Ring A, Ring B, linker L, R¹, R^{2a}, R^{2b}, R³ and R⁴ are as defined above.

In various embodiments, the definition of groups is as defined above.

In one embodiments, the compounds of Formula (I) is selected from the group consisting of:
N-(4-(4-Nitrophenoxy)phenyl)-2-oxo-3-(propan-2-ylidene)indoline-5-sulfonamide,
2-oxo-N-(4-(phenylamino)phenyl)-3-(propan-2-ylidene)indoline-5-sulfonamide,
N-(4-((2-oxo-3-(propan-2-ylidene)indoline)-5-sulfonamido)phenyl)benzamide,
N-(4-benzoylphenyl)-2-oxo-3-(propan-2-ylidene)indoline-5-sulfonamide,
2-oxo-N-(5-phenyl-1H-pyrazol-3-yl)-3-(propan-2-ylidene)indoline-5-sulfonamide,
2-oxo-3-(propan-2-ylidene)-N-(5-(p-tolyl)-1H-pyrazol-3-yl)indoline-5-sulfonamide,
2-oxo-N-(5-phenyl-1,3,4-thiadiazol-2-yl)-3-(propan-2-ylidene)indoline-5-sulfonamide,
N-(benzo[d]thiazol-2-yl)-2-oxo-3-(propan-2-ylidene)indoline-5-sulfonamide,
N-(4-(1H-imidazol-1-yl)phenyl)-2-oxo-3-(propan-2-ylidene)indoline-5-sulfonamide,
N-methyl-4-(4-((2-oxo-3-(propan-2-ylidene)indoline)-5-sulfonamido)phenoxy)picolinamide,
4-(3-fluoro-4-((2-oxo-3-(propan-2-ylidene)indoline)-5-sulfonamido)phenoxy)-N-methylpicolinamide,
2-fluoro-N-methyl-4-((2-oxo-3-(propan-2-ylidene)indoline)-5-sulfonamido)benzamide,
N-(4-methyl-3-((4-(pyridin-3-yl)pyrimidin-2-yl)amino)phenyl)-2-oxo-3-(propan-2-ylidene)indoline-5-sulfonamide, and
N-(1,4-dioxo-1,2,3,4-tetrahydrophthalazin-5-yl)-2-oxo-3-(propan-2-ylidene)indoline-5-sulfonamide,
   or a pharmaceutically acceptable salt, stereoisomers, enantiomers, prodrugs, hydrates or solvates thereof.

The compounds of Formula (I) can be prepared according to various chemical synthesis methods. In one embodiment, the compounds can be prepared via Scheme 1 depicted below:

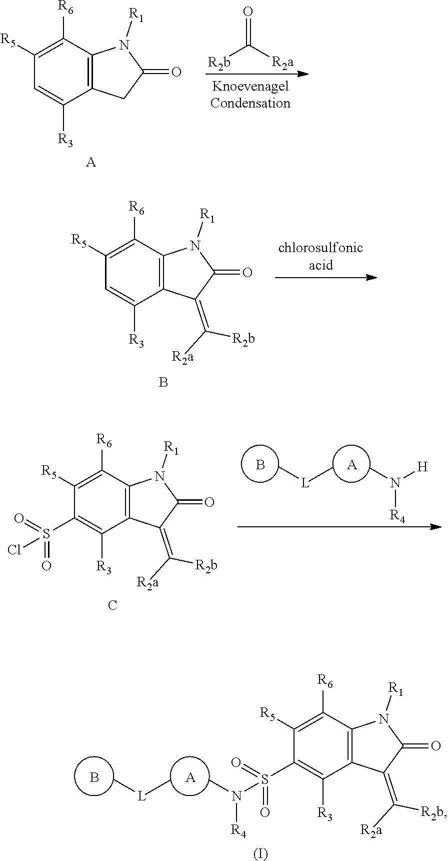

wherein R¹, R^{2a}, R^{2b}, R³, R⁴, R⁵, R⁶, Ring A, Ring B and L are as defined above.

The compounds described herein can be prepared pharmaceutical compositions by methods which are commonly used, using excipients commonly used in this field, that is, pharmaceutical excipients, pharmaceutical carrier, or the like.

As a solid composition for the oral administration, a tablet, powder, a granule, and the like is used. In such solid composition, one or two or more kinds of active ingredients are mixed with at least one inert excipient. The composition may contain an inert additive, for example, a lubricant, a disintegrant, a stabilizer, a solubilizer, and the like by commonly used methods.

A liquid composition for the oral administration includes an emulsion, a solution preparation, a suspension, a syrup or an elixir, and the like which is pharmaceutically acceptable, and includes a generally used inert diluent, for example, purified water or ethanol. The liquid composition may contain adjuvants such as a solubilizing agent, a wetting agent, and a suspension, a sweetener, a flavor, an aromatic, or a preservative in addition to the inert diluent.

The solid composition or the liquid composition may also be encapsulated to form capsules, including but not being limited to soft capsules or hard capsules.

The pharmaceutical composition may also be used via injection to a subject. The injection for the parenteral administration includes a sterile aqueous or non-aqueous solution preparation, a suspension or an emulsion. As the aqueous solvent, for example, distilled water for injection or physiological saline is included. As the non-aqueous solvent, for example, alcohols such as ethanol are included. Such a composition may further include a tonicity agent, a preservative, a wetting agent, an emulsifier, a dispersant, a stabilizer, or a solubilizer. These are sterilized by, for example, filtration through a bacteria-retaining filter, mixing of a germicide, or irradiation. In addition, these can also be used in a manner in which a sterile solid composition is prepared, and is dissolved or suspended in sterile water or a sterile solvent for injection before being used.

Transmucosal agent such as a transnasal agent and the like is used in a solid, liquid, or semi-solid form, and can be prepared according to methods known in the related art. For example, a known excipient, a pH adjuster, a preservative, a surfactant, a lubricant, a stabilizer, a thickener, and the like may be suitably added. In administration, it is possible to use an appropriate device for inhalation or insufflation.

In another aspect, the present disclosure is directed to a method of preventing or treating an inflammatory condition or fibrosis disease, which comprises administering a therapeutically effective amount of the compound or a pharmaceutically acceptable salt, stereoisomers, enantiomers, prodrugs, hydrates and solvates thereof as defined herein to a subject in need thereof.

Example inflammatory conditions or fibrosis disease that may be treated using CDK8 inhibitors include, but are not limited to, Type 1 diabetes graft-versus-host disease, inflammatory bowel disease, psoriasis, psoriatic arthritis, Hashimoto's thyroiditis, food allergy, HCV vasculitis, alopecia areata, systemic lupus erythematosus, multiple sclerosis, rheumatoid arthritis; skin fibrosis, lung fibrosis, renal fibrosis, liver fibrosis, intestinal fibrosis, cystic fibrosis, carciac fibrosis, uterine leiomyoma and adenomyosis.

In certain embodiments, the lung fibrosis is idiopathic pulmonary fibrosis (IPF).

In another aspect, the present disclosure is directed to a method of preventing or treating tumor or cell proliferative disease, which comprises administering a therapeutically effective amount of the compound or a pharmaceutically acceptable salt, stereoisomers, enantiomers, prodrugs, hydrates and solvates thereof to a subject in need thereof

In certain embodiments, the tumor or cell proliferative disease or cancer metastasis is at least one selected from the group consisting of epithelial cancer, epidermoid cancer, Merkel cell carcinoma, liver cancer, cervical cancer, anal cancer, penile cancer, vulvar cancer, vaginal cancer, breast cancer, ovarian cancer, uterine cancer, skin cancer, melanoma, oral cancer, colon cancer, neck cancer, real cancer, kidney cancer, liver cancer, colorectal cancer, oesophageal cancer, thyroid cancer, uveal cancer, acute myelogenous, acute myeloid leukemia, head cancer, eye cancer, chronic myelogneous leukemia, myeloid cell leukemia, glioma, Kaposi's sarcoma or human laryngeal squamous cell carbinoma, leukemia, nasopharyngeal carcinoma, mesothelioma, bone cancer, brain cancer, prostate cancer, testicular cancer, pancreatic cancer, hepatocellular carcinoma, lung cancer, squamous cancer, and lymphoma. In some embodiments, the cancer is identified as overexpressing CDK8. In certain embodiments, the cancer is colon cancer, breast cancer and prostate cancer (CRC).

The invention having now been described by way of written description, those of skill in the art will recognize that the invention can be practiced in a variety of embodiments and that the foregoing description and examples below are for purposes of illustration and not limitation of the claims that follow.

### EXAMPLE

### Materials and Methods

### Materials

The primary antibodies against N-Cadherin, Snail, p-Smad2 (Ser465/467), Smad3, p-RNA Pol II (Ser2/5), Akt, p-Akt (Ser473), p-GSK3β (Ser9), active form β-catenin, histone3, and labelled secondary antibodies anti-rabbit IgG-HRP were purchased from Cell Signaling Technology (Danvers, MA, USA). The primary antibodies against STAT1, p-STAT1 (Ser727) were from Abcam (Cambridge, MA, USA), while those against E-Cadherin, vimentin, p-Smad3 (Thr179), β-actin were from ABclonal (Woburn, MA, USA). CDK8, β-SMA, GSK3β-tubulin primary antibodies and anti-rabbit IgG secondary antibody DyLight 594 were purchased from GeneTex Inc. (Hsinchu, Taiwan). The pcDNA3 CDK8 HA (P #634) was a gift from Matija Peterlin (Addgene plasmid #14649; http://n2t. net/addgene: 14649; RRID:Addgene_14649); 7TFP CDH1 reporter was a gift from Bob Weinberg (Addgene plasmid #91704; http://n2t. net/addgene: 91704; RRID:Addgene_91704). TurboFect transfection reagent was from Thermo Fisher Scientific (Waltham, MA, USA).

### Kinase Inhibition Assay

Compounds were tested for kinase inhibitory activity using the Thermo Fisher SelectScreen service (www.thermofisher.com/selectscreen). For CDK8, the LanthaScreen Eu Binding Assay was used. Briefly, the assay employed utilizes a fluorescent tracer to a given kinase. This is used to detect the addition of an anti-tag antibody. When the tracer and antibody bind to the targeted kinase, the Forster resonance energy transfer is measured. When a kinase inhibitor is added to the assay, the tracer/kinase binding will be disrupted, producing low FRET detection and indicating kinase inhibitory activity. Evaluation of kinase activity of additional kinase targets utilized the LanthaScreen binding assay or the Z′-Lyte activity assay provided by Thermo Fisher SelectScreen service. The IC50 values were determined using sequential doses, and the inhibition curves were provided by Thermo Fisher Scientific.

### Cell Culture

Human A549 alveolar epithelial cells, human prostate cancer cell lines PC-3 and DU145were obtained from the Bioresource Collection and Research Center (Hsinchu City, Taiwan). The human A549 alveolar epithelial cells were maintained in Dulbecco's modified Eagle medium (DMEM; Invitrogen Life Technologies, USA) supplemented with 10% fetal bovine serum (v/v) (Invitrogen Life Technologies, USA), penicillin (100 units/mL), and streptomycin (100 μg/mL) (Biological Industries, Israel). The human prostate cancer cell lines PC-3 and DU145 cultured in Roswell Park Memorial Institute (RPMI) 1640 medium or Minimum essential medium Eagle supplemented with 10% fetal bovine serum (v/v), penicillin (100 units/mL) and streptomycin (100 μg/mL). All cells were maintained at 37° C. in a humidified atmosphere with 5% CO₂.

### Cell Cytotoxicity Assay

### Human A549 Alveolar Epithelial Cells

Cell cytotoxicity was measured by a colorimetric MTT assay. Cells (104) in 1 mL of medium in 96-well plates were incubated with the vehicle (control) or vehicle with test compound for 48 h. After various treatments, 1 mg/mL of MTT was added, and the plates were incubated at 37° C. for an additional 2 h. Then cells were pelleted and lysed with 10% sodium dodecylsulfate (SDS) with 0.01 M HCl, and the absorbance at 570 nm was measured on a microplate reader.

### Human Prostate Cancer Cell Lines PC-3 and DU145

Cell cytotoxicity was evaluated using the MTT assay. Cells in 100 μL medium in 96-well plates were treated with vehicle or test compound for 24 h. Then, 0.5 mg/mL MTT was added, and the plates were incubated for an additional 2 h at 37° C. The cells were then pelleted and lysed in 100 μL of dimethyl sulfoxide, and the absorbance at 550 nm was measured with a microplate reader.

### RNA Extraction and Real-Time Polymerase Chain Reaction (PCR)

### Human A549 Alveolar Epithelial Cells

Total RNA was isolated using TRIzol and a Direct-zol™ RNA MiniPrep kit (ZYMO Research, USA) following the manufacturer's instructions. Reverse-transcription to complementary (c) DNA was performed using a random primer and M-MLRT. In brief, first-strand cDNA was synthesized using 1 μg of messenger (m) RNA incubated with a random primer at 65. C for 5 min and then reacted with M-MLRT at 37° C. for 1 h. For the real-time PCR, cDNAs were amplified in SYBR Green PCR Master Mix (Life Technologies, USA) and detected with the Applied Biosystems StepOnePlus™ Q-PCR detection system (Thermo Fisher Scientific, USA). Relative gene expression was normalized to GAPDH and calculated by using the 2^{(-ΔΔCT)} method.

### Human Prostate Cancer Cell Lines PC-3 and DU145

Total RNA was isolated from cells using TRIzol reagent (Invitrogen). Single-strand cDNA for a polymerase chain reaction template was synthesized from 5 μg of total RNA using random primers and Moloney murine leukemia virus reverse transcriptase (Promega). Quantitative PCR was performed using TaqMan One Step RT-PCR mastermix (ABI) in a total reaction volume of 20 μL per reaction, consisting of 10 μL of SYBR green PCR master mix (Applied Biosystems), 5 pmol of each forward/reverse primer, and 2 μL of cDNA. The oligonucleotide primers used for the amplification were as follows: for E-cadherin, 5′-AAAGGCCCATTTCCTAAAAACCT-3′ (forward) and 5′-TGCGTTCTCTATCCAGAGGCT-3′ (reverse); Snail, 5′-GAGGACAGTGGGAAAGGCTC-3′ (forward) and 5′-TGGCTTCGGATGTGCATCTT-3′ (reverse); α-SMA, 5′-AAAAGACAGCTACGTGGGTGA-3′ (forward) and 5′-GCCATGTTCTATCGGGTACTTC 3′ (reverse); GAPDH, 5′-CCATCACCATCTTCCAGGAGCG-3′ (forward) and 5′-AGAGATGATGACCCTTTTGGC-3′ (reverse). GAPDH served as the endogenous control to normalize variations in total RNA levels in each sample. The threshold cycle (Ct) in the exponential phase of amplification was detected, the relative mRNA expression level was determined by calculating the ΔΔCt values, and the fold change was expressed as 2^{-ΔΔCt}. The value of each control sample was set at 1 and was used to calculate the fold change in the expression of target genes.

### Immunoblot and Immunoprecipitation Analyses

### Human A549 Alveolar Epithelial Cells

Cells (106) were incubated for 10 min at 4° C. in lysis buffer (20 mM HEPES at pH 7.4, 2 mM EGTA, 50 mM β-glycerophosphate, 0.1% Triton X-100, 10% glycerol, 1 mM DTT, 1 μg/mL of leupeptin, 5 μg/mL of aprotinin, 1 mM phenylmethylsulfonyl fluoride, and 1 mM sodium orthovanadate), scraped off, incubated on ice for an additional 10 min, and centrifuged at 17,000 g for 30 min at 4° C. Protein samples (20 μg) were then electrophoresed on SDS-polyacrylamide gel electrophoresis (PAGE) gels and transferred to a nitrocellulose membrane, which was then blocked by incubation for 30 min at room temperature with 5% bovine serum albumin (BSA) in Tris-buffered saline with 0.1% Tween-20 (TBST). Immunoblotting was performed by overnight incubation at 4° C. with primary antibodies in TBST, followed by incubation for 1 h at room temperature with horseradish peroxidase (HRP)-conjugated secondary antibodies. Bound antibodies were measured using enhanced chemiluminescence (ECL) reagent (GE Healthcare, UK) and exposed to photographic film.

### Human Prostate Cancer Cell Lines PC-3 and DU145

The cells were incubated in lysis buffer for 10 min at 4° C., scraped off, incubated on ice for an additional 10 min and then centrifuged at 17,000 g for 30 min at 4° C. Protein samples were electrophoresed on SDS-PAGE gels, transferred to PVDF membranes and blocked with 5% fat-free milk in PBS for 30 min at room temperature. Immunoblotting was performed overnight with primary antibodies in PBS at 4° C., followed by incubation with HRP-conjugated secondary antibodies for 1 h at room temperature. Bound antibodies will be measured using an ECL reagent and exposed to a photographic film. In the immunoprecipitation assay, cell lysates were immunoprecipitated with 1 μg of antibody and A/G agarose beads overnight at 4° C. After washing the precipitated beads three times with 1 mL ice-cold cell lysis buffer, bound immune complexes were separated by 10% SDS-PAGE and analyzed with immunoblotting.

### Immunofluorescence Staining

### Human Prostate Cancer Cell Lines PC-3 and DU145

Cells were seeded into 24-well plates, treated with drugs for 12 h, and fixed with 8% paraformaldehyde in PBS for 15 min. After washing twice with PBS (10 min/wash), the cells were permeabilized with 0.1% Triton X-100 in PBS for 10 min. The cells were rinsed twice with PBS for 10 min, blocked with 5% BSA in PBS for 1 h, and then incubated with a primary antibody overnight, followed by a 2 h incubation with FITC-conjugated anti-rabbit IgG antibody. The mounting medium, which contained DAPI stain, was dropped onto the slides, and cover slides were recovered to the slides. Images were detected by a ZEISS ApoTome.2 microscope.

### Wound-Healing Assay

### Human A549 Alveolar Epithelial Cells

Cells were seeded in six-well plates and cultured to 90% confluency, at which time the cellular monolayer was wounded with a sterile 10-μL pipette tip and washed with culture medium to remove detached cells. Cells were incubated in the presence or absence of test compounds for 24 h and photographed. The wound closure percentages were subsequently determined using ImageJ software (National Institutes of Health, USA).

### Cellular Dissection Method

### Human A549 Alveolar Epithelial Cells

A Nuclear/Cytosol Fractionation Kit (Biovision, USA) was used to separate the cytosolic and nuclear fractions. Briefly, cells were collected and centrifuged at 600 g for 5 min, supernatants were removed, and the lysate was resuspended in Cytosol Extraction Buffer-A, vortexed vigorously for 15 s, and placed on ice for 10 min. Cytosol Extraction Buffer-B was then added to the mixture, vortexed for 5 s, incubated on ice for 1 min, and centrifuged at 14,500 rpm to acquire the cytosolic fraction. The remaining pellets were resuspended in nuclear extraction buffer, vortexed for 15 s, and then iced for 10 min. After repeating this procedure four times, samples were centrifuged at 14,500 rpm to acquire the nuclear extract.

### Estimation of Oxidative Stress

### Human A549 Alveolar Epithelial Cells

An end product of lipid peroxidation, malondialdehyde (MDA), was measured by spectrophotometry at a wavelength of 532 nm using malondialdehyde bis(dimethyl acetal) as the standard as per the assay protocol (Cell Biolabs, USA).

### Immunohistochemical Analysis

Human prostate cancer cell lines PC-3 and DU145

Tissue sections underwent dewaxing and rehydration. Antigen retrieval was performed by autoclaving the slides in Trilogy solution (Cell Marque, Hot Springs, AR) at 121° C. for 10 min. The slides were blocked with 3% H₂O₂ and 5% fetal bovine serum, incubated with primary antibodies at 4° C. overnight, and then treated with polymer-HRP reagent (Dako Cytomation, Glostrup, Denmark). activity Peroxidase was visualized using diamino-benzidine tetrahydrochloride solution (DAKO), and sections were counterstained with hematoxylin. Dark brown nuclear staining was defined as positive, no staining was defined as negative.

### Data Analysis and Statistical Analysis

Data are expressed as the mean±standard error of the mean (SEM) and were analyzed using a one-way analysis of variance (ANOVA). When the ANOVA showed significant differences between groups, Tukey's post hoc test was used to determine the pairs of groups showing statistically significant differences. Parameters with p<0.05 were considered statistically significant.

For differential expression analysis of individual genes, two online database repositories, cBioPortal (www.cbioportal.org) and Gene Expression Omnibus (GEO; www.ncbi.nlm.nih.gov/geo/) were utilized to access clinical transcriptomic datasets. The datasets (TCGA, n=494; GSE74685, n=171) were downloaded and analyzed for mRNA levels of the queried genes across different prostate cancer patient groups. Computation of statistical significance as well as the construction of box plots were performed using SigmaPlot software. Kaplan-Meier graphs were generated with the KM plotter online tool (www.kmplot.com). Datasets obtained from cBioPortal and the GEO database that contained information on the time until cancer progression (TCGA, n=492) and biochemical relapse (GSE70769, n=92) were transferred onto the site for curve plotting. Gene expression cutoffs to define the low and high expression groups were determined by Cox proportional hazards regression analysis, and the best-performing cutoff was selected for the final analysis. The expression patterns of entire signal pathways or protein complexes were evaluated via Gene Set Enrichment Analysis (GSEA) software version 4.2.2. Patient samples in datasets acquired from cBioPortal (Abida et al.) and the GEO database (GSE68882, GSE16560) were first manually sorted into two groups (according to Gleason score or metastasis event) and then imported into the program for gene ranking.

### Synthetic Procedures

### General Procedures for Synthesizing Compounds a-n

Nuclear magnetic resonance (NMR) spectra (1H and ¹³C NMR) were obtained using Bruker Fourier 300 MHZ, JEOL 400 MHZ, A VIII 500 MHZ, and Agilent 600 MHz spectrometers with standard plus programs. Chemical shifts are presented in parts per million (ppm, 8) with TMS as an internal standard. The mass spectroscopic (MS) data were measured on a Finnigan Mat TSQ-7000 mass spectrometer (HRESIMS). HPLC was performed using a C18 column (250×4.6 mm, Waters) and an L-2130 (Hitachi, Japan)/LC-20AT (Shimadzu, Japan) pump. Column chromatography was performed on silica gel (70-230 mesh, Merck, Germany). The thin layer chromatographic (TLC) analysis was performed on silica gel plates (KG60-F254, Merck). Unless otherwise mentioned, all chemicals and materials were used as received from commercial suppliers without no purification. Anhydrous dichloromethane was distilled from calcium hydride under N₂.

### Example 1: Synthesis of Compound Library (Compound a to n)

An exemplified general Route for Example 1 is shown below:

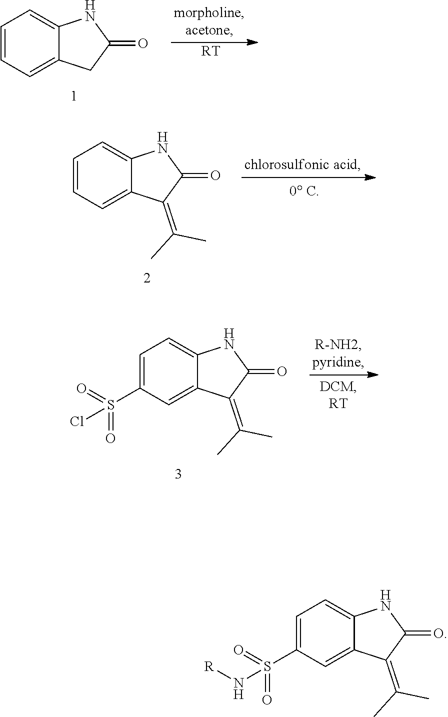

### Preparation of 3-(Propan-2-ylidene)indolin-2-one (Compound 2)

To a stirred solution of compound 1 (100 mg, 0.75 mmol) in CH₃H₆O (3 mL, 0.25 M) was added morpholine (65 mg, 0.75 mmol). The solution was stirred at reflux temperature for 22 h, and then concentrated in vacuo. The residue was diluted with distilled H₂O. The solid was collected by filtration and washed with distilled H₂O to give compound 2 (154 mg, 89%) as a solid. 1H NMR (300 MHz, CDCl₃) δ 8.22 (s, 1H), 7.52 (d, J=7.6 Hz, 1H), 7.19 (m, 1H), 7.01 (m, 1H), 6.86 (d, J=7.8 Hz, 1H), 2.62 (s, 3H), 2.38 (s, 3H). ¹³C NMR (125 MHz, CDCl₃) & 169.9, 155.7, 139.5, 127.7, 124.5, 123.8, 123.2, 121.7, 109.5, 25.4, 23.3

### Preparation of 2-Oxo-3-(propan-2-ylidene)indoline-5-sulfonyl chloride (Compound 3)

Chlorosulfonic acid (269 mg, 2.31 mmol) was slowly added dropwise to compound 2 (100 mg, 0.58 mmol). The solution was stirred at an ice-bath for 2 h. Following the procedure as described for compound 2 gave 3 (153 mg, 98%) as a solid. 1H NMR (300 MHz, CDCl₃) δ 8.33 (s, 1H), 8.14 (d, J=1.3 Hz, 1H), 7.93 (dd, J=1.9, 8.4 Hz, 1H), 7.03 (d, J=8.4 Hz, 1H), 2.68 (s, 3H), 2.50 (s, 3H). ¹³C NMR (125 MHz, DMSO-d₆) δ 168.8, 161.2, 147.4, 137.2, 128.9, 125.7, 122.7, 121.9, 110.4, 25.5, 23.2

The compounds described herein, e.g., the following exemplary compounds, can be synthesized in analogy to the procedures as described above with commercially available and proper starting materials or intermediates prepared as herein described.

### Preparation of N-(4-(4-Nitrophenoxy)phenyl)-2-oxo-3-(propan-2-ylidene)indoline-5-sulfonamide (Compound a)

To a solution of compound 3 (100 mg, 0.37 mmol) in THF-H₂O (5:3, 16 mL) was added K₂CO₃ (102 mg, 0.74 mmol) and 4-(4-nitrophenoxy) aniline (85 mg, 0.37 mmol). The resulting solution was stirred at room temperature (RT) for 5 h, and then concentrated in vacuo. The residue was diluted with distilled H₂O (10 mL), neutralized with 1 N HCl_{(aq)} to pH 7, and extracted with EtOAc (30 mL×3). The organic layer was dried over Na₂SO₄ and then removed in vacuo after being filtered. The residue was purified by silica gel chromatography (MeOH/CH₂Cl₂=1:99) to give compound a (146 mg, 40%) as a solid. 1H NMR (300 MHz, DMSO-d₆) δ 10.90 (s, 1H, H-1), 10.19 (s, 1H, H-9), 8.15 (m, 2H, H-19, 21), 7.82 (s, 1H, H-4), 7.58 (dd, J=1.3, 8.3 Hz, 1H, H-6), 7.17 (d, J=8.9 Hz, 2H, H-11, 15), 7.08 (d, J=8.9 Hz, 2H, H-12, H-14), 7.01 (m, 2H, H-18, 22), 6.93 (d, J=8.2 Hz, 1H, H-7), 2.53 (s, 3H, H-1′), 2.30 (s, 3H, H-3′). ¹³C NMR (125 MHz, DMSO-d₆) δ 168.3 (C-2), 165.0 (C-20), 162.9 (C-2′), 158.0 (C-13), 143.9 (C-7a), 142.1 (C-17), 135.3 (C-10), 133.7 (C-5), 127.2 (C-6), 126.1 (C-19, 21), 123.7 (C-4a), 122.6 (C-11, 15), 121.5 (C-3, 4), 121.4 (C-12, 14), 117.0 (C-18, 22), 108.8 (C-7), 24.9 (C-3′), 22.6 (C-1′). HRMS-ESI: m/z [M+H]⁺ calcd. for C₂₃H₂₀O₆N₃S 466.1067, found 466.1070.

### Preparation of 2-Oxo-N-(4-(phenylamino)phenyl)-3-(propan-2-ylidene)indoline-5-sulfonamide (Compound b)

To a stirred solution of N1-phenylbenzene-1,4-diamine (68 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 3 h, and then concentrated in vacuo. Following the procedure as described for compound a gave b (159 mg, 99%) as a solid. 1H NMR (600 MHz, DMSO-d₆) δ 10.84 (s, 1H, H-1), 9.67 (s, 1H, H-9), 8.03 (s, 1H, H-16), 7.72 (m, 1H, H-4), 7.54 (dd, J=1.8, 8.2 Hz, 1H, H-6), 7.18 (m, 2H, H-19, 21), 6.95 (dd, J=1.1, 8.6 Hz, 2H, H-18, 22), 6.93 (m, 4H, H-11, 12, 14, 15), 6.90 (d, J=8.2 Hz, 1H, H-7), 6.77 (m, 1H, H-20), 2.51 (s, 3H, H-1′), 2.26 (s, 3H, H-3′). ¹³C NMR (125 MHZ, DMSO-d₆) δ 168.3 (C-2), 157.6 (C-2′), 143.6 (C-7a), 143.5 (C-17), 140.5 (C-10), 131.6 (C-5), 129.7 (C-13), 129.1 (C-14, 19, 21), 127.1 (C-4a, 6), 123.5 (C-11, 15), 121.6 (C-3), 121.5 (C-4), 119.4 (C-20), 117.5 (C-12), 116.2 (C-18, 22), 108.7 (C-7), 24.9 (C-3′), 22.5 (C-1′). HRMS-ESI: m/z [M+H]⁺ calcd. for C₂₃H₂₂O₃N₃S 420.1376, found 420.1377.

### Preparation of N-(4-((2-Oxo-3-(propan-2-ylidene)indoline)-5-sulfonamido) phenyl)benzamide (Compound c)

To a stirred solution of N-(4-aminophenyl)benzamide (78 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 5 h, and then concentrated in vacuo. Following the procedure as described for compound a gave c (169 mg, 99%) as a solid. 1H NMR (600 MHz, DMSO-d₆) δ 10.85 (s, 1H, H-1), 10.16 (s, 1H, H-16), 10.00 (s, 1H, H-9), 7.90 (m, 2H, H-19, 23), 7.82 (m, 1H, H-4), 7.61 (d, J=8.8 Hz, 2H, H-11, 15), 7.57 (m, 2H, H-6, 21), 7.50 (t, J=7.5 Hz, 2H, H-20, 22), 7.07 (m, 2H, H-12, 14), 6.90 (d, J=8.2 Hz, 1H, H-7), 2.52 (s, 3H, H-1′), 2.30 (s, 3H, H-3′). ¹³C NMR (125 MHZ, DMSO-d₆) δ 168.3 (C-2), 165.3 (C-17), 157.8 (C-2′), 143.7 (C-7a), 135.7 (C-18), 134.8 (C-10), 133.4 (C-5), 131.5 (C-13), 131.4 (C-21), 128.3 (C-20, 22), 127.5 (C-19, 23), 127.2 (C-6), 123.6 (C-4a), 121.5 (C-3), 121.4 (C-4), 121.2 (C-11, 15), 121.1 (C-12, 14), 108.8 (C-7), 24.9 (C-3′), 22.5 (C-1′). HRMS-ESI: m/z [M+H]⁺ calcd. for C₂₄H₂₂O₄N₃S 448.1326, found 448.1325.

### Preparation of N-(4-Benzoylphenyl)-2-oxo-3-(propan-2-ylidene)indoline-5-sulfonamide (Compound d)

To a stirred solution of (4-aminophenyl) (phenyl) methanone (58 mg, 0.29 mmol) in anhydrous CH₂Cl₂ (10 mL) was sequentially added pyridine (47 mg, 0.59 mmol) and compound 3 (100 mg, 0.37 mmol). The solution was stirred at RT under N₂ for 6 h, and then concentrated in vacuo. Following the procedure as described for compound a gave d (137 mg, 86%) as a solid. ¹H NMR (600 MHZ, DMSO-d₆) δ 10.90 (s, 1H, H-1), 10.76 (s, 1H, H-9), 7.91 (m, 1H, H-4), 7.71 (dd, J=1.8, 8.2 Hz, 1H, H-6), 7.65 (d, J=8.7 Hz, 2H, H-12, 14), 7.64 (dt, J=1.8, 3.2 Hz, 1H, H-20), 7.63 (q, J=1.8 Hz, 2H, H-18, 22), 7.52 (m, 2H, H-19, 21), 7.28 (m, 2H, H-11, 15), 6.95 (d, J=8.2 Hz, 1H, H-7), 2.52 (s, 3H, H-1′), 2.32 (s, 3H, H-3′). ¹³C NMR (125 MHZ, DMSO-d₆) δ 194.4 (C-16), 168.3 (C-2), 158.3 (C-2′), 144.1 (C-7a), 142.3 (C-10), 137.3 (C-17), 132.3 (C-13), 131.6 (C-5), 131.4 (C-12, 14), 131.3 (C-20), 129.3 (C-19, 21), 128.4 (C-18, 22), 127.3 (C-6), 123.8 (C-4a), 121.4 (C-3), 121.3 (C-4), 118.0 (C-14, 15), 109.0 (C-7), 24.9 (C-3′), 22.6 (C-1′). HRMS-ESI: m/z [M+H]⁺ calcd. for C₂₄H₂₁O₄N₂S 433.1217, found 433.1212.

### Preparation of 2-Oxo-N-(5-phenyl-1H-pyrazol-3-yl)-3-(propan-2-ylidene)indoline-5-sulfonamide (Compound e)

To a stirred solution of 5-phenyl-1H-pyrazol-3-amine (59 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 4 h, and then concentrated in vacuo. Following the procedure as described for compound a, the residue was purified by silica gel chromatography (MeOH/CH₂Cl₂=1:33) to give e (112 mg, 30%) as a solid. 1H NMR (600 MHZ, DMSO-d₆) δ 12.81 (s, 1H, H-12), 10.84 (s, 1H, H-1), 10.32 (s, 1H, H-9), 7.91 (s, 1H, H-4), 7.67 (t, J=1.5 Hz, 2H, H-16, 20), 7.66 (m, 2H, H-17, 19), 7.42 (t, J=7.5 Hz, 1H, H-6), 7.34 (d, J=7.3 Hz, 1H, H-18), 6.92 (d, J=8.2 Hz, 1H, H-7), 6.44 (s, 1H, H-14), 2.51 (s, 3H, H-1′), 2.27 (s, 3H, H-3′). ¹³C NMR (125 MHZ, DMSO-d₆) δ 168.4 (C-2), 157.5 (C-2′), 147.0 (C-10), 143.7 (C-7a), 142.5 (C-13), 132.3 (C-5), 128.9 (C-18), 128.3 (C-15), 127.1 (C-6, 17, 19), 124.9 (C-16, 20), 123.5 (C-4a), 121.7 (C-21), 121.5 (C-3), 108.7 (C-7), 94.6 (C-14), 24.8 (C-3′), 22.5 (C-1′). HRMS-ESI: m/z [M+H]⁺ calcd. for C₂₀H₁₉O₃N₄S 395.1172, found 395.1171.

### Preparation of 2-Oxo-3-(propan-2-ylidene)-N-(5-(p-tolyl)-1H-pyrazol-3-yl) indoline-5-sulfonamide (Compound f)

To a stirred solution of 5-(p-tolyl) 1H-pyrazol-3-amine (64 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 6 h, and then concentrated in vacuo. The solid was collected by filtration and washed with CH₂Cl₂ to give compound f (91 mg, 61%) as a solid. 1H NMR (300 MHz, DMSO-d₆) δ 12.72 (s, 1H, H-12), 10.85 (s, 1H, H-1), 10.31 (s, 1H, H-9), 7.90 (s, 1H, H-4), 7.65 (dd, J=1.6, 8.2 Hz, 1H, H-6), 7.54 (d, J=8.1 Hz, 2H, H-16, 20), 7.22 (d, J=8.0 Hz, 2H, H-17, 19), 6.92 (d, J=8.2 Hz, 1H, H-7), 6.36 (s, 1H, H-14), 2.49 (s, 3H, H-1′), 2.31 (s, 3H, H-21), 2.27 (s, 3H, H-3′). ¹³C NMR (125 MHZ, DMSO-d₆) δ 168.4 (C-2), 157.5 (C-2′), 147.0 (C-10), 143.7 (C-7a), 142.7 (C-13), 137.7 (C-18), 132.3 (C-5), 129.5 (C-15, 17, 19), 127.1 (C-6), 124.9 (C-16, 20), 123.5 (C-4a), 121.7 (C-3), 121.5 (C-4), 108.7 (C-7), 94.2 (C-14), 24.8 (C-3′), 22.5 (C-1′), 20.8 (C-21). HRMS-ESI: m/z [M+H]⁺ calcd. for C₂₁H₂₁O₃N₄S 409.1329, found 409.1325.

### Preparation of 2-Oxo-N-(5-Phenyl-1,3,4-thiadiazol-2-yl)-3-(propan-2-ylidene) indoline-5-sulfonamide (Compound g)

To a stirred solution of 5-phenyl-1,3,4-thiadiazol-2-amine (65 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 6 h, and then concentrated in vacuo. Following the procedure as described for compound f gave g (95 mg, 26%) as a solid. 1H NMR (300 MHz, DMSO-d₆) δ 10.88 (s, 1H, H-1), 10.48 (s, 1H, H-9), 7.91 (d, J=1.4 Hz, 1H, H-4), 7.82 (m, 1H, H-17), 7.70 (dd, J=1.7, 8.3 Hz, 1H, H-6), 7.55 (m, 2H, H-18, 19), 7.45 (dd, J=1.4, 8.0 Hz, 1H, H-16), 6.97 (d, J=8.3 Hz, 1H, H-7), 6.75 (d, J=8.0 Hz, 1H, H-20), 52 (s, 3H, H-1′), 2.32 (s, 3H, H-3′). ¹³C NMR (125 MHz, DMSO-d₆) δ 168.7 (C-2), 168.4 (C-10), 157.8 (C-2′), 155.2 (C-15), 143.7 (C-7a), 141.5 (C-13), 134.1 (C-5), 131.4 (C-18), 129.4 (C-17), 126.3 (C-19), 126.2 (C-6), 125.4 (C-16), 123.6 (C-4a), 121.5 (C-3), 120.6 (C-4), 109.1 (C-7), 107.8 (C-20), 24.7 (C-3′), 22.2 (C-1′). HRMS-ESI: m/z [M+H]⁺ calcd. for C₁₉H₁₅O₃N₄S₂ 411.0580, found 411.0590.

### Preparation of N-(Benzo[d]thiazol-2-yl)-2-oxo-3-(propan-2-ylidene)indoline-5-sulfonamide (Compound h)

To a stirred solution of benzo[d]thiazol-2-amine (55 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 24 h, and then concentrated in vacuo. Following the procedure as described for compound f gave h (84 mg, 59%) as a solid. 1H NMR (300 MHz, DMSO-d₆) δ 10.48 (s, 1H, H-1), 7.87 (d, J=7.9 Hz, 1H, H-4), 7.78 (s, 1H, H-6), 7.46 (m, 2H, H-13, 14), 7.42 (m, 1H, H-15), 7.29 (t, J=7.5 Hz, 1H, H-12), 6.75 (d, J=7.9 Hz, 1H, H-7), 2.50 (s, 3H, H-1′), 2.31 (s, 3H, H-3′). ¹³C NMR (125 MHZ, DMSO-d₆) δ 168.9 (C-2), 168.7 (C-10), 154.4 (C-2′), 140.4 (C-7a), 134.2 (C-5), 127.4 (C-15), 125.3 (C-13), 124.0 (C-12), 123.5 (C-4a), 122.9 (C-4), 122.6 (C-3, 12a), 122.5 (C-15a), 120.9 (C-6), 114.5 (C-14), 107.9 (C-7), 24.7 (C-3′), 22.2 (C-1′). HRMS-ESI: m/z [M+H]⁺ calcd. for C₁₈H₁₄O₃N₃S₂ 384.0471, found 384.0482.

### Preparation of N-(4-(1H-imidazol-1-yl)phenyl)-2-oxo-3-(propan-2-ylidene) indoline-5-sulfonamide (Compound i)

To a stirred solution of 4-(1H-imidazol-1-yl) aniline (41 mg, 0.26 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 20 h, and then concentrated in vacuo. Following the procedure as described for compound f gave i (100 mg, 68%) as a solid. 1H NMR (600 MHz, DMSO-d₆) δ 10.90 (s, 1H, H-1), 10.51 (s, 1H, H-9), 8.91 (s, 1H, H-17), 7.90 (d, J=10.6 Hz, 2H, H-19, 20), 7.64 (dd, J=1.6, 8.2 Hz, 1H, H-6), 7.58 (m, 2H, H-12, 14), 7.50 (s, 1H, H-4), 7.27 (dd, J=2.5, 9.4 Hz, 2H, H-11, 15), 6.93 (d, J=8.2 Hz, 1H, H-7), 2.52 (s, 3H, H-1′), 2.32 (s, 3H, H-3′). ¹³C NMR (125 MHZ, DMSO-d₆) δ 168.3 (C-2), 158.2 (C-2′), 144.0 (C-7a), 138.0 (C-10), 134.8 (C-17), 131.2 (C-5), 127.3 (C-6), 124.8 (C-13), 123.8 (C-4a), 122.4 (C-12, 14), 121.4 (C-19), 121.3 (C-3), 121.0 (C-4), 120.7 (C-11, 15), 119.6 (C-20), 108.9 (C-7), 25.0 (C-3′), 22.6 (C-1′). HRMS-ESI: m/z [M+H]⁺ calcd. for C₂₀H₁₉O₃N₄S 395.1172, found 395.1169.

### Preparation of N-Methyl-4-(4-((2-oxo-3-(propan-2-ylidene)indoline)-5-sulfonamido)phenoxy)picolinamide (Compound j)

To a stirred solution of 4-(4-aminophenoxy)-N-methylpicolinamide (90 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 2 h, and then concentrated in vacuo. Following the procedure as described for compound a gave j (174 mg, 97%) as a solid. 1H NMR (600 MHZ, DMSO-d₆) δ 10.88 (s, 1H, H-1), 10.21 (s, 1H, H-9), 8.73 (m, 1H, H-24), 8.47 (d, J=5.7 Hz, 1H, H-21), 7.84 (s, 1H, H-4), 7.58 (dd, J=1.7, 8.1 Hz, 1H, H-6), 7.31 (d, J=2.3 Hz, 1H, H-18), 7.20 (m, 2H, H-11, 15), 7.12 (m, 2H, H-12, 14), 7.04 (dd, J=5.7, 2.6 Hz, 1H, H-22), 6.94 (d, J=8.1 Hz, 1H, H-7), 2.78 (d, J=4.8 Hz, 3H, H-25), 2.53 (s, 3H, H-1′), 2.31 (s, 3H, H-3′). ¹³C NMR (125 MHz, DMSO-d₆) δ 168.3 (C-2), 165.5 (C-19), 163.7 (C-23), 158.0 (C-2′), 152.5 (C-17), 150.4 (C-21), 149.6 (C-13), 143.9 (C-7a), 135.6 (C-10), 131.3 (C-5), 127.2 (C-6), 123.7 (C-4a), 122.6 (C-11, 15), 121.8 (C-12, 14), 121.4 (C-3, 4), 114.0 (C-22), 108.9 (C-7), 108.8 (C-18), 26.0 (C-25), 24.9 (C-3′), 22.5 (C-1′). HRMS-ESI: m/z [M+H]⁺ calcd. for C₂₄H₂₃O₅N₄S 479.1384, found 479.1382.

### Preparation of 4-(3-Fluoro-4-((2-oxo-3-(propan-2-ylidene)indoline)-5-sulfonamido)phenoxy)-N-methylpicolinamide (Compound k)

To a stirred solution of 4-(4-amino-3-fluorophenoxy)-N-methylpicolinamide (96 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 6 h, and then concentrated in vacuo. Following the procedure as described for compound a gave k (178 mg, 97%) as a solid. 1H NMR (500 MHZ, DMSO-d₆) δ 10.92 (s, 1H, H-1), 10.10 (s, 1H, H-9), 8.78 (q, J=4.7 Hz, 1H, H-24), 8.52 (d, J=5.5 Hz, 1H, H-21), 7.84 (d, J=1.3 Hz, 1H, H-4), 7.54 (dd, J=1.7, 8.2 Hz, 1H, H-6), 7.38 (d, J=2.5 Hz, 1H, H-18), 7.34 (t, J=8.8 Hz, 1H, H-15), 7.22 (dd, J=2.7, 10.9 Hz, 1H, H-12), 7.13 (dd, J=2.7, 5.5 Hz, 1H, H-22), 7.04 (m, 1H, H-14), 6.95 (d, J=8.2 Hz, 1H, H-7), 2.79 (d, J=4.9 Hz, 3H, H-25), 2.54 (s, 3H, H-1′), 2.31 (s, 3H, H-3′). ¹³C NMR (125 MHZ, DMSO-d₆) δ 168.4 (C-2), 164.9 (C-19), 163.6 (C-23), 158.0 (C-2′), 157.3 (C-10), 155.3 (C-11), 152.5 (C-17), 151.6 (C-13), 150.5 (C-21), 143.9 (C-7a), 131.7 (C-6), 128.4 (C-15), 127.1 (C-7), 123.7 (C-5), 122.3 (C-12), 121.5 (C-3), 121.4 (C-4), 117.1 (C-14), 114.4 (C-22), 109.3 (C-18), 108.8 (C-7), 26.0 (C-25), 24.9 (C-3′), 22.5 (C-1′). HRMS-ESI: m/z [M+H]⁺ calcd. for C₂₄H₂₂O₅N₄FS 497.1289, found 497.1290.

### Preparation of 2-Fluoro-N-methyl-4-((2-oxo-3-(propan-2-ylidene)indoline)-5-sulfonamido)benzamide (Compound l)

To a stirred solution of 4-amino-2-fluoro-N-methylbenzamide (62 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 4 h, and then concentrated in vacuo. Following the procedure as described for compound a gave l (120 mg, 81%) as a solid. 1H NMR (300 MHz, DMSO-d₆) δ 10.91 (s, 1H, H-1), 10.75 (s, 1H, H-9), 8.01 (m, 1H, H-17), 7.89 (s, 1H, H-4), 7.68 (dd, J=1.7, 8.2 Hz, 1H, H-6), 7.51 (t, J=8.5 Hz, 1H, H-12), 6.97 (m, 2H, H-11, 15), 6.92 (m, 1H, H-7), 2.71 (d, J=4.6 Hz, 3H, H-18), 2.52 (s, 3H, H-1′), 2.33 (s, 3H, H-3′). ¹³C NMR (125 MHZ, DMSO-d₆) δ 168.3 (C-2), 163.3 (C-16), 160.4 (C-13), 158.4 (C-2′), 144.2 (C-7a), 141.7 (C-14), 133.2 (C-5), 131.3 (C-10), 130.9 (C-12), 127.3 (C-6), 123.9 (C-4a), 121.4 (C-3), 121.3 (C-4), 114.4 (C-11), 109.0 (C-7), 105.7 (C-15), 26.2 (C-18), 25.0 (C-3′), 22.6 (C-1′). HRMS-ESI: m/z [M+H]⁺ calcd. for C₁₉H₁₉O₄N₃FS 404.1075, found 404.1074.

### Preparation of N-(4-Methyl-3-((4-(pyridin-3-yl)pyrimidin-2-yl)amino)phenyl)-2-oxo-3-(propan-2-ylidene)indoline-5-sulfonamide (Compound m)

To a stirred solution of 6-methyl-N1-(4-(pyridin-3-yl)pyrimidin-2-yl)benzene-1,3-diamine (102 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 3 h, and then concentrated in vacuo. Following the procedure as described for compound f gave m (64 mg, 34%) as a solid. 1H NMR (300 MHz, DMSO-d₆) δ 10.80 (s, 1H, H-1), 10.05 (s, 1H, H-9), 9.24 (d, J=1.5 Hz, 1H, H-25), 8.87 (s, 1H, H-17), 8.72 (dd, J=1.5, 4.9 Hz, 1H, H-22), 8.49 (m, 1H, H-29), 8.46 (m, 1H, H-27), 7.80 (s, 1H, H-4), 7.60 (m, 2H, H-6, 28), 7.44 (m, 2H, H-11, 21), 7.07 (d, J=8.4 Hz, 1H, H-14), 6.88 (d, J=8.2 Hz, 1H, H-7), 6.81 (dd, J=2.3, 8.1 Hz, 1H, H-15), 2.45 (s, 3H, H-1′), 2.17 (s, 3H, H-3′), 2.12 (s, 3H, H-16). ¹³C NMR (125 MHZ, DMSO-d₆) δ 168.3 (C-2), 160.9 (C-18), 160.8 (C-20), 159.5 (C-27), 157.6 (C-2′), 149.7 (C-22), 146.6 (C-25), 143.7 (C-7a), 138.1 (C-12), 136.1 (C-29), 135.9 (C-10), 132.7 (C-24), 131.6 (C-5), 130.6 (C-14), 127.6 (C-13), 127.1 (C-6), 124.5 (C-28), 123.5 (C-4a), 121.6 (C-4), 121.4 (C-3), 116.7 (C-11), 116.4 (C-15), 108.9 (C-7), 107.8 (C-21), 24.7 (C-3′), 22.5 (C-1′), 17.4 (C-16). HRMS-ESI: m/z [M+H]⁺ calcd. for C₂₇H₂₅O₃N₆S 513.1703, found 513.1703.

### Preparation of N-(1,4-Dioxo-1,2,3,4-tetrahydrophthalazin-5-yl)-2-oxo-3-(propan-2-ylidene)indoline-5-sulfonamide (Compound n)

To a stirred solution of 5-Amino-2,3-dihydro-1,4-phthalazinedione (73 mg, 0.37 mmol) in anhydrous CH₂Cl₂ (10 mL) was sequentially added pyridine (58 mg, 0.74 mmol) and compound 3 (100 mg, 0.37 mmol). The solution was stirred at RT under N₂ for 5 h, and then concentrated in vacuo. Following the procedure as described for compound a, the residue was purified by silica gel chromatography (MeOH/CH₂Cl₂=1:20-1:5) to give n (25 mg, 17%) as a solid. 1H NMR (400 MHZ, DMSO-d₆) δ 13.06 (s, 1H, H-9), 11.79 (s, 2H, H-15, 16), 10.83 (s, 1H, H-1), 7.84 (d, J=1.6 Hz, 1H, H-4), 7.77 (m, 2H, H-11, 12), 7.68 (dd, J=1.6, 8.0 Hz, 1H, H-6), 7.54 (dd, J=2.8, 6.4 Hz, 1H, H-13), 6.92 (d, J=8.0 Hz, 1H, H-7), 2.49 (s, 3H, H-1′), 2.28 (s, 3H, H-3′). ¹³C NMR (100 MHZ, DMSO-d₆) δ 168.0 (C-2), 160.3 (C-17), 158.0, (C-2′), 151.8 (C-14), 144.1 (C-7a), 140.2 (C-10), 134.3 (C-12), 130.9 (C-5), 127.2 (C-6), 126.3 (C-14a), 123.7 (C-4a), 121.2 (C-3, 4), 119.2 (C-11), 117.9 (C-13), 114.8 (C-17a), 109.0 (C-7), 24.6 (C-3′), 22.4 (C-1′). HRMS-ESI: m/z [M+H]⁺ calcd. for C₁₉H₁₇O₅N₄S 413.0914, found 413.0911.

### Example 2. Evaluation of Synthesized Compounds

In this study, a fragment-based drug design strategy was used to optimize the reference CDK8 inhibitor E966-0539, N-(3-acetylphenyl)-2-oxo-3-(propan-2-ylidene)-indoline-5-sulfonamide, previously discovered through a structure-based virtual screening (SBVS) campaign. The 50% inhibitory concentration (IC50) value of E966-0530 in an enzymatic assay was 1,684 nM. The E966-0530 structure contains an indolin-2-one backbone with a sulfonamide group. Sulfonamide is a key functional group in organic chemistry with high hydrolytic stability and capacity for amino acid interactions (A. Ovung et al, 2021 Biophys. Rev. 13 (2) 259-272). Indolin-2-one is one of the privileged core skeletons that continue to emerge in natural products and pharmacologically active compounds (Y. M. Khetmalis et al, 2021 Biomed. Pharmacother. 141, 111842). The above experiments and analyses indicated that E966-0530 was a promising starting point for further optimization. The optimization included designing and synthesizing a series of 2-oxo-3-(propan-2-ylidene)indoline-5-sulfonamide derivatives.

The fragment-based drug design strategy in this study led to optimized CDK8 inhibitors with promising results. CDK8-inhibitory activities of the compounds (a-n) designed by this fragment-based drug design strategy were tested using the SelectScreen kinase service of ThermoFisher Scientific. The inhibitory effects of the 14 compounds and the referenced compound were tested at a concentration of 10 μM. Results are shown in Table 1 below:

TABLE 1

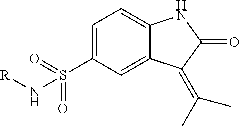

| Compound | | CDK8 Inhibition | Inhibition of CDK8 |
|---|---|---|---|
| No. | Structure of R | percentage at 10 μM | IC₅₀ (nM) |
| a | 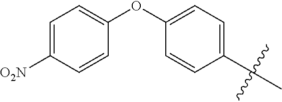 [SMILES: *c1ccc(Oc2ccc([N+](=O)[O-])cc2)cc1] | 48% | Not determined |
| b | 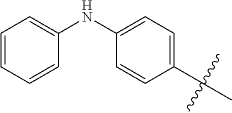 [SMILES: *c1ccc(Nc2ccccc2)cc1] | 76% | 1567 |
| c | 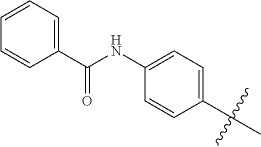 [SMILES: *c1ccc(NC(=O)c2ccccc2)cc1] | 33% | Not determined |
| d | 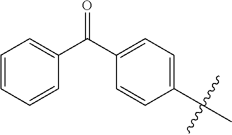 [SMILES: *c1ccc(C(=O)c2ccccc2)cc1] | 99% | 380 |
| e | 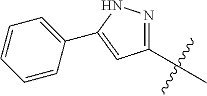 [SMILES: *c1cc(-c2ccccc2)[nH]n1] | 82% | 1639 |
| f | 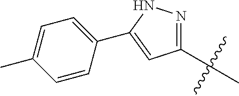 [SMILES: *c1cc(-c2ccc(C)cc2)[nH]n1] | 89% | 798 |
| g | 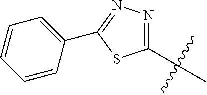 [SMILES: *c1nnc(-c2ccccc2)s1] | 99% | 338 |
| h | 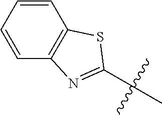 [SMILES: *c1nc2ccccc2s1] | 91% | 870 |
| i | 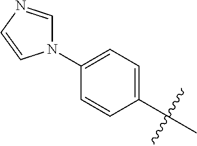 [SMILES: *c1ccc(-n2ccnc2)cc1] | 92% | 945 |
| j | 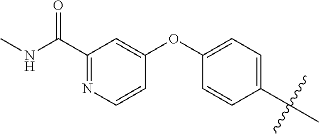 [SMILES: *c1ccc(Oc2ccnc(C(=O)NC)c2)cc1] | 85% | 1235 |
| k | 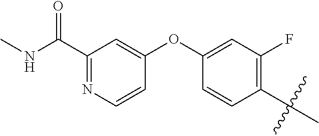 [SMILES: *c1ccc(Oc2ccnc(C(=O)NC)c2)cc1F] | 102% | 129 |
| 1 | 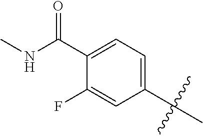 [SMILES: *c1ccc(C(=O)NC)c(F)c1] | 92% | 652 |
| m | 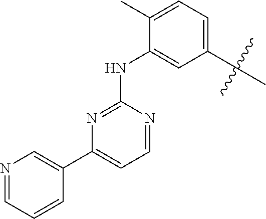 [SMILES: *c1ccc(C)c(Nc2nccc(-c3cccnc3)n2)c1] | 46% | Not determined |
| n | 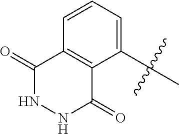 [SMILES: *c1cccc2c(=O)[nH][nH]c(=O)c12] | 74% | 3004 |
| Reference Compound | 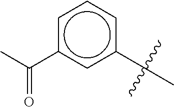 | 87% | 1684 |

As can be seen from Table 1, results showed that 11 compounds exhibited ≥50% inhibition. Further testing showed the compounds exhibiting IC50 values between 129 and 3,004 nM. Seven compounds showed IC50 values within the hundreds of nM range. Notably, three of 5 the compounds inhibited CDK8 with IC50 values of <400 nM. Those compounds were d, g, and k with respective IC50 values of 380, 338, and 129 nM. Compound k showed a 13-fold increase in CDK8 inhibitory activity compared to the original compound E966-0530. Experimental results showed that compound k was the most potent among the synthesized compounds.

### Example 3. Docking and SAR Analysis of Compounds a-n

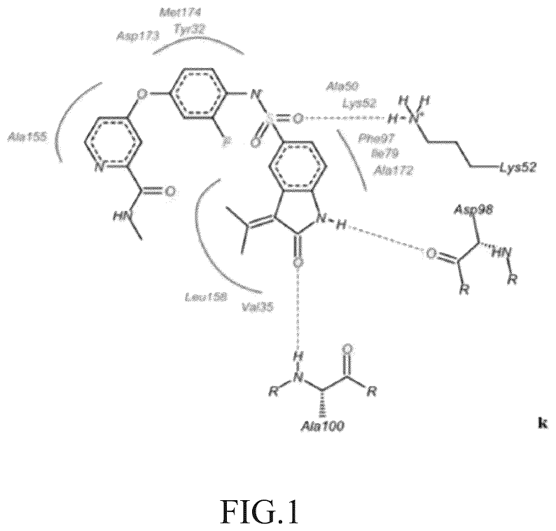

FIG. 1 shows the 2D interaction pose of compound k.

To investigate interactions that may promote CDK8 inhibition, compound k was molecularly docked into the CDK8 binding site (FIG. 1).

The structure of compound k was divided into three groups (G1, G2, and G3). The docking results indicated that the oxindole ring of the G1 group occupied the adenine position and formed hydrogen bonds with residues D98 and A100 of hinge residues. Produced by nitrogen and oxygen atoms located on the oxindole ring, these interactions were identified as binding interactions. An additional pi-pi T-shaped interaction with residue F97 was also observed within the G1 group. The G2 group was a sulfonamide structure. Residue K52 formed hydrogen bonds with oxygen atoms and formed an attractive charge with amines. Finally, the G3 group was 4-(4-amino-3-fluorophenoxy)-N-methylpicolinamide. A pi-alkyl interaction occurred between residue M174 and the aromatic ring of phenoxypyridine. An additional halogen interaction of the fluorine atom with residue N156 was also observed. Overall, these interactions suggest that compound k exhibited favorable interactions against CDK8.

Further, a SAR analysis was performed using compounds a-n. Together, the analysis showed favorable functional groups and interactions that may be important for potent CDK8 inhibition.

First, compound j, which has a single moiety difference compared to compound k, was then selected for analysis. From the inhibitory activity, compound k was found to have a nearly 1.5-fold higher inhibitory potency due to the substitution of halogen atoms. In addition, the effect of the compounds e-i with the amine group at the C5-C6 or C6-C5 was explored. The composition of C5 included pyrazole, thiadiazole, and thiazole. Among them, compound g composed of thiadiazole showed good inhibitory activity and was also the second most effective in this series of compounds. The docking analysis revealed that compound g formed pi-anion interactions with residue K52 by its sulfonic amide and thiadiazole, which may play a key role in CDK8 inhibition.

Compounds a-d were obtained by connecting diphenyl structures with different linking atoms to sulfonamides. Among them, compound d exhibited inhibitory activity with an IC50 value of 380 nM. From the structural analysis, it was found that benzophenone of compound d formed additional hydrophobic interactions with residues V27, V35, Y99, and W105, and the compound had better inhibitory activity.

### Example 4. Inhibition of CDK8 Activity with Remarkable Selectivity

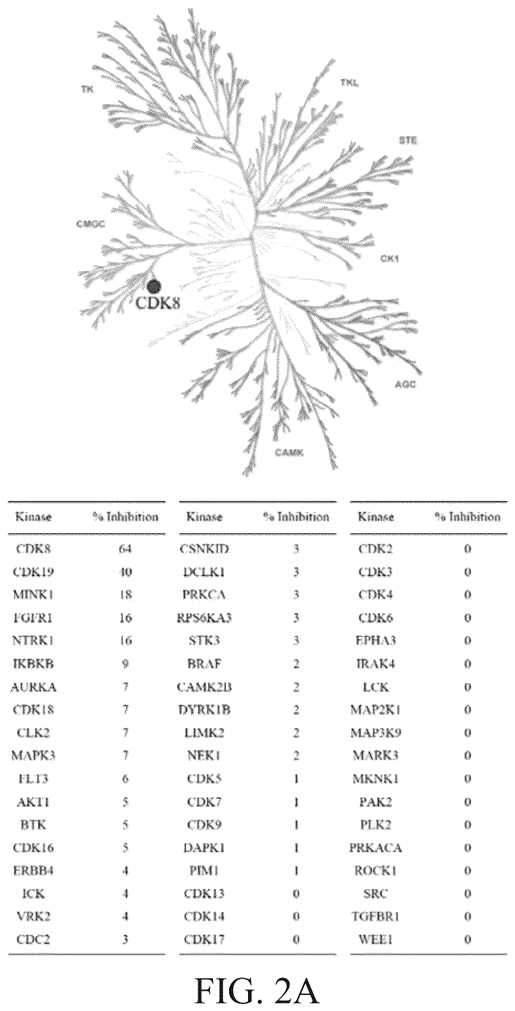

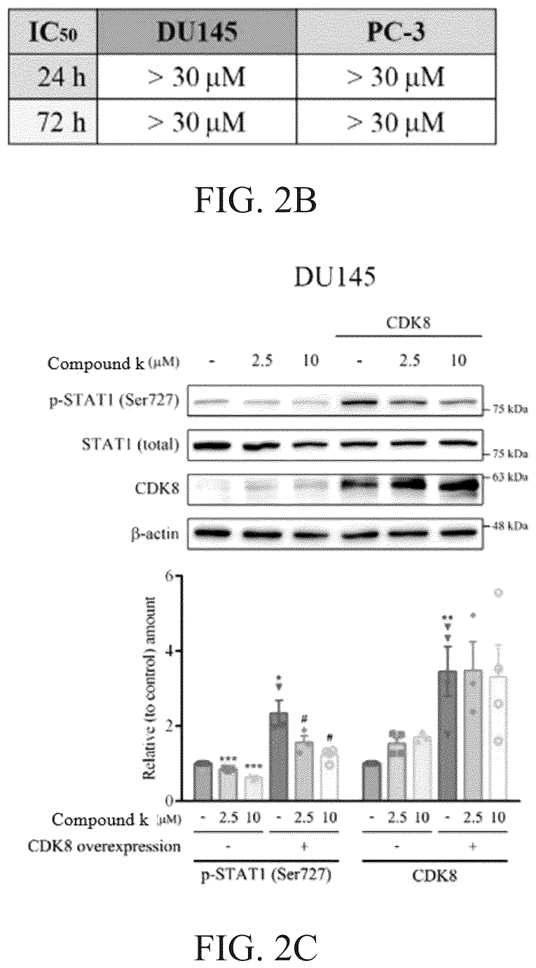

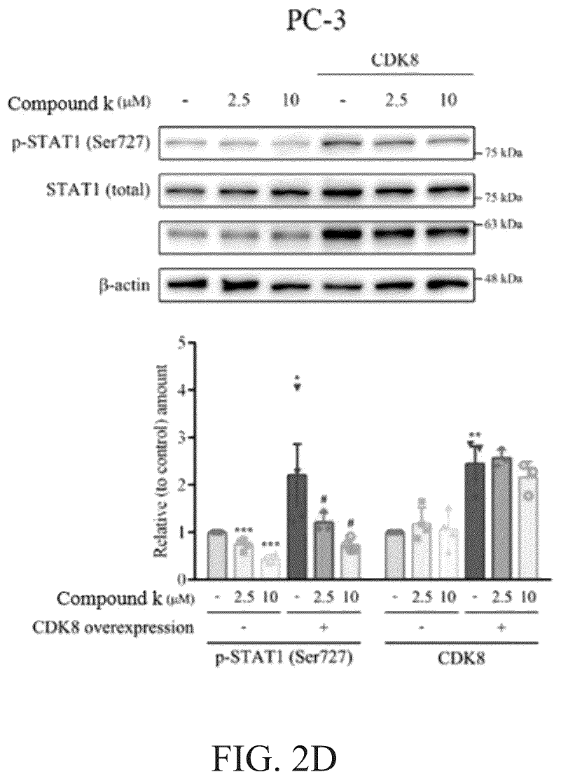

FIGS. 2A-2D show the evaluation of CDK8 inhibition by compound k.

Compound k was evaluated for kinase selectivity. A panel of 54 kinases from different families was selected, and the kinase inhibition of compound k was screened at 150 nM. As shown in FIG. 2A, among all the kinases, only CDK8 was inhibited by compound k with an inhibition percentage of >60% (64%). The inhibition of CDK19, a paralog of CDK8, by compound k was slightly weaker than that of CDK8 (40%), and the compound had no significant effect on other CDKs and kinases. Higher selectivity of the compound might reduce the risk of adverse side effects, and the selectivity of compound k may enhance its safety and avoid adverse side effects. The cytotoxicity of compound k was then assessed in DU145 and PC-3 cells, human prostate cancer cell lines isolated from patients with brain and bone metastasis, respectively. As shown in FIG. 2B, the IC50 of compound k was more than 30 μM for 24 h in both cell lines and sustained for 72 h. These results showed that compound k administered at concentrations below 30 μM from 24 h to 72 h did not cause significant cytotoxicity in either cell line. To further examine whether compound k still inhibited CDK8/19 inhibition in cells, we assessed whether compound treatment in DU145/PC-3 androgen-independent prostate cancer cells could inhibit STAT1-S727 phosphorylation. Since STAT1 is a substrate of CDK8/19, STAT1-S727 phosphorylation is commonly used to measure CDK8/19 kinase activity (J. Bancerek et al, 2013 Immunity 38 (2) 250-262). As shown, compound k treatment suppressed the phosphorylation of the known CDK8 substrate STAT1 at the Ser727 residue in a concentration-dependent manner (FIGS. 2C-2D). To further confirm the CDK8-inhibitory effects of the compound, cells were transfected with the pcDNA3 CDK8 HA plasmid to overexpress CDK8. Compound k significantly inhibited the phosphorylation of STAT1 at Ser727 in a concentration-dependent manner without affecting CDK8 expression in both cell lines (FIGS. 2C-2D). These results indicated that compound k can inhibit CDK8/19 activity in cells.

### Example 5. CDK8 Expression and Transforming Growth Factor β TGF-β) Signals were Increased During Prostate Cancer Progression

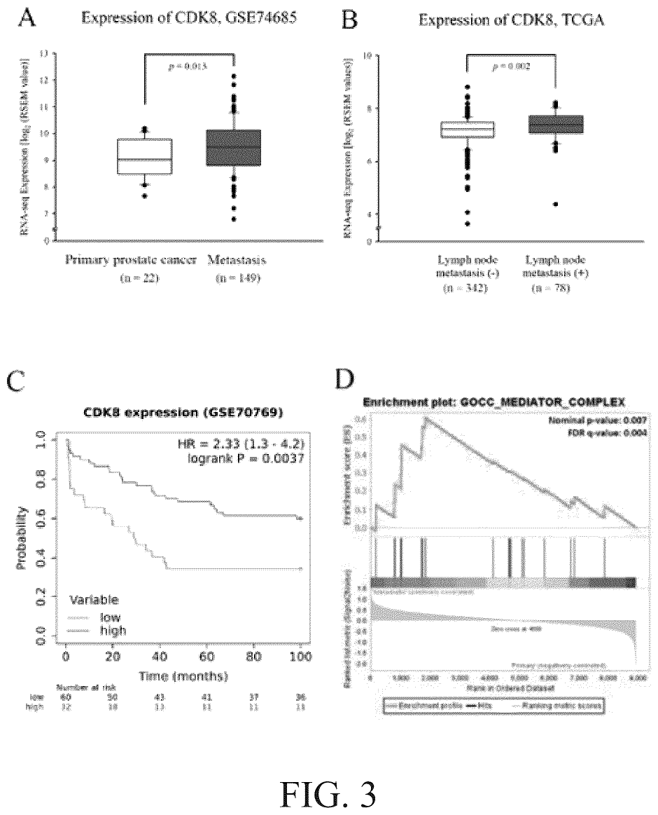

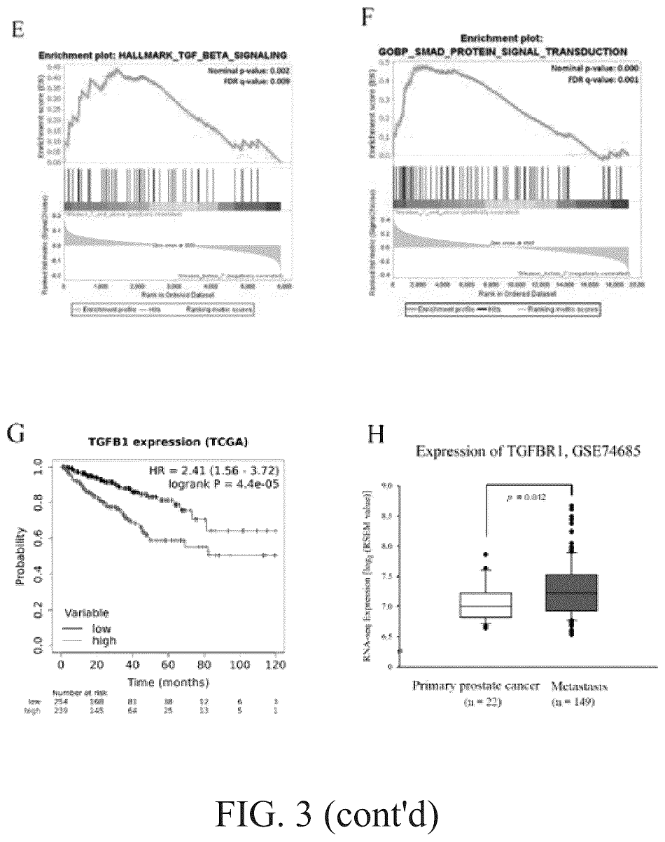

FIGS. 3A-3H show that CDK8 expression and TGF-β signals are increased in metastatic prostate cancer and are associated with poor patient survival.

To examine the relationship between CDK8 and prostate cancer progression, public datasets for clinical samples were accessed via the GEO database and cBioPortal. As shown, higher CDK8 RNA expression was found in metastatic prostatic cancer samples than in primary prostate cancer samples (FIG. 3A), and similar tendencies were noted in a separate series when the samples were sorted according to the presence of lymph node metastasis (FIG. 3B). Expression array data collected from 94 radical prostatectomy patient samples were used to analyze the time until relapse, and high CDK8 RNA levels were found to correlate significantly with earlier biochemical relapse over a 100-month period (FIG. 3C). Given the CDK8's affiliation with the mediator complex, gene set enrichment analysis (GSEA) was conducted in an exploratory attempt to profile the expression of the multiprotein complex as a whole (GSE68882). The analysis revealed that components of the mediator complex, including CDK8/19, MED12, and MED13, were also upregulated in metastatic prostate cancer (FIG. 3D). These observed trends echoed the findings of previous studies and prompted us to explore causal models that may explain such correlations (I. B. Roninson et al, 2019 Cells 8 (8) 821; J. Bragelmann et al, 2017 Clin. Cancer Res 23 (7) 1829-1840). Additionally, TGF-β is a prominent inducer of cancer metastasis, and previous research has shown that overproduction of TGF-β is linked to metastasis, androgen receptor inhibition resistance, and poor clinical outcomes in prostate cancer (T. Trivedi et al, 2021 Biomolecules 11 (11) 1643). Furthermore, the Gleason score is a grading system used to determine the aggressiveness of prostate cancer. A score higher than 7 indicates cancers that are likely to spread more rapidly. Using Gleason score 7 (intermediate grade) as a cutoff point, changes in TGF-β/Smad signals were analyzed. Database enrichment analyses indicated enhanced TGF-β/Smad signaling in samples with high pathological Gleason scores (FIG. 3E, 3 F). High TGF-β/Smad signal expression was linked to worse 10-year progression-free survival (FIG. 3G), and high TGFBR1 transcript levels were observed in metastatic prostate cancer samples (FIG. 3H). These results suggested that the TGF-β/Smad pathway plays a pivotal role in prostate cancer metastasis, and the CDK8/mediator complex might be a very important regulator of this signal.

### Example 6. Inhibition on the Cell Migration and the Expression of EMT Proteins

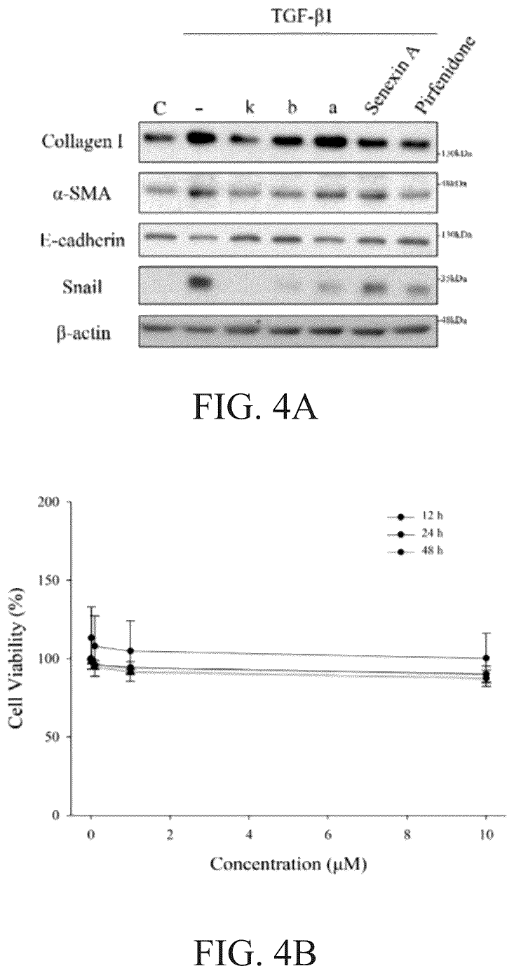

FIGS. 4A-4B show the inhibition of epithelial-to-mesenchymal transition protein expressions by CDK8 inhibitors in human alveolar epithelial cells.

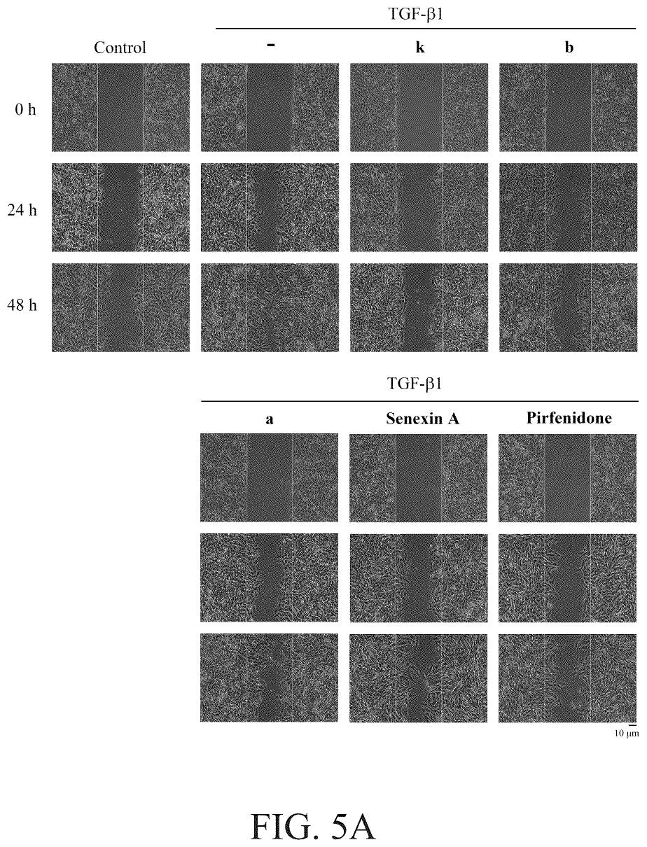

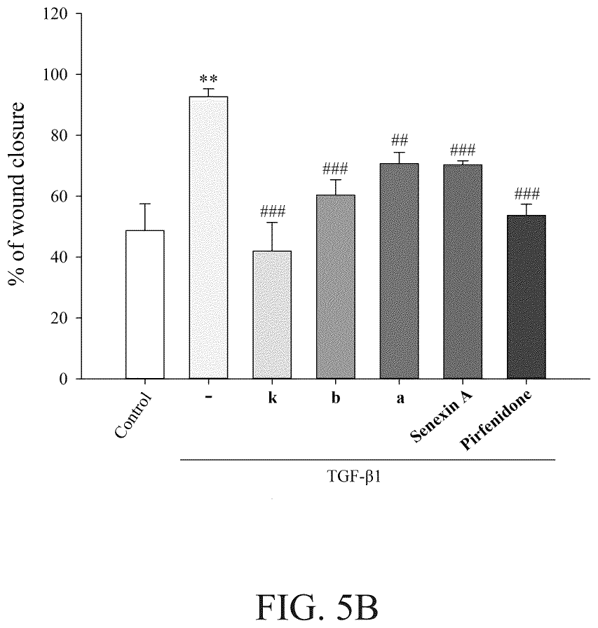

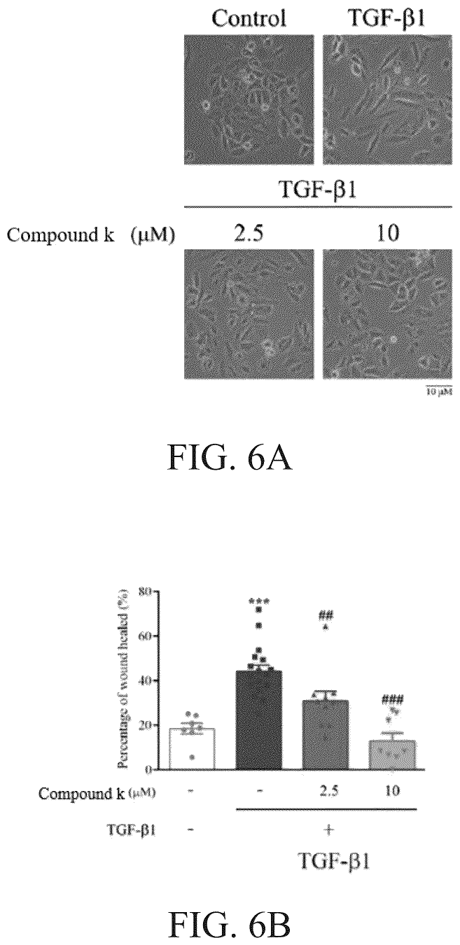

FIGS. 5A-5B show that compound k inhibited the migration of A549 cells.

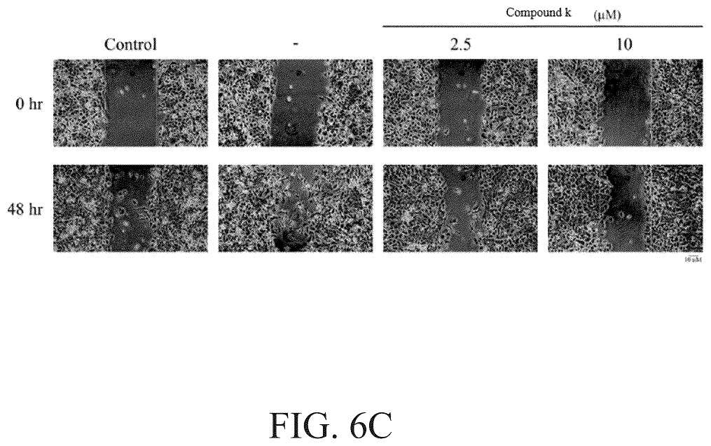

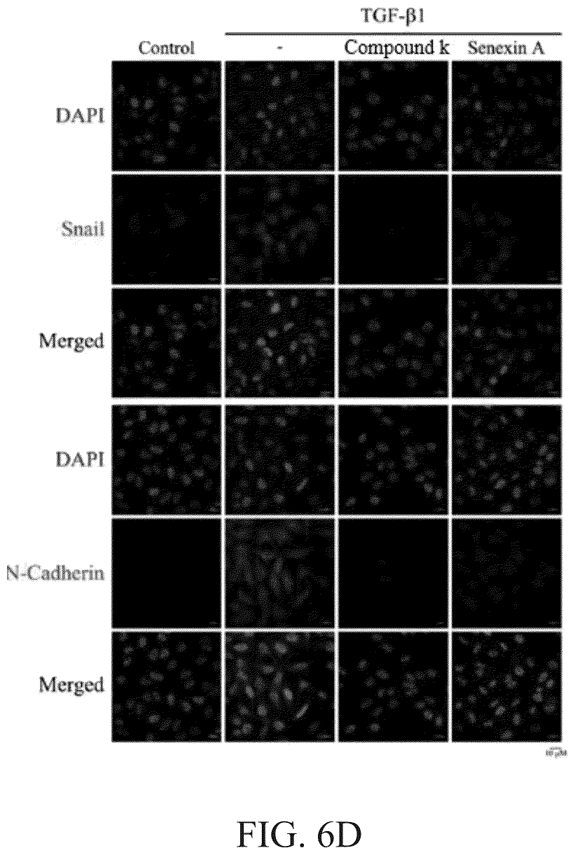

FIGS. 6A-6D show that cell migration and EMT features of prostate cancer cells were notably suppressed by compound k.

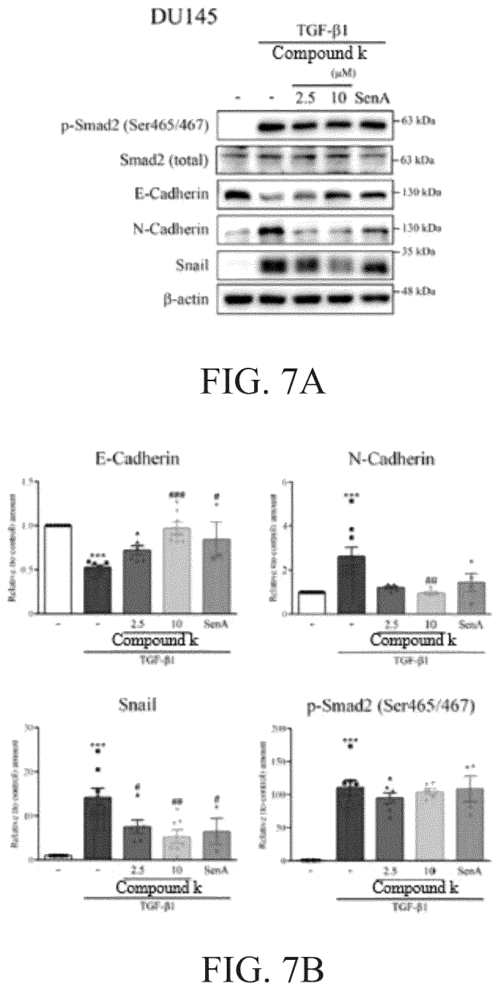

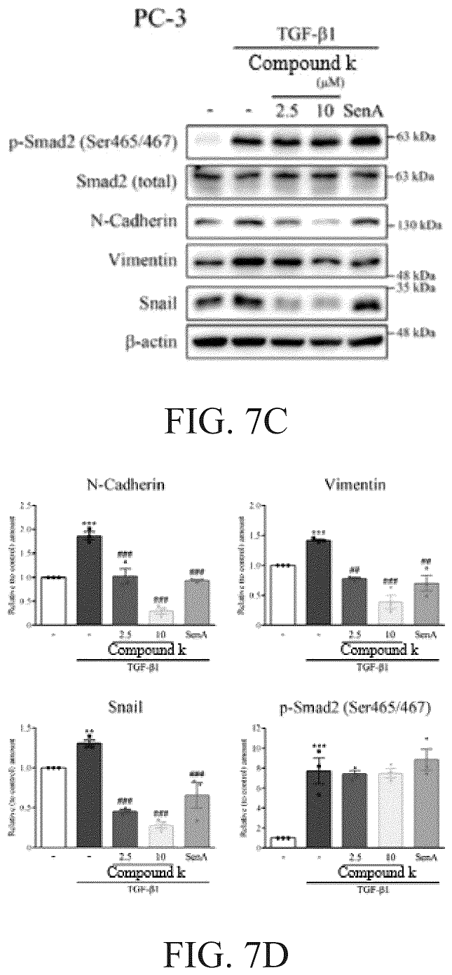

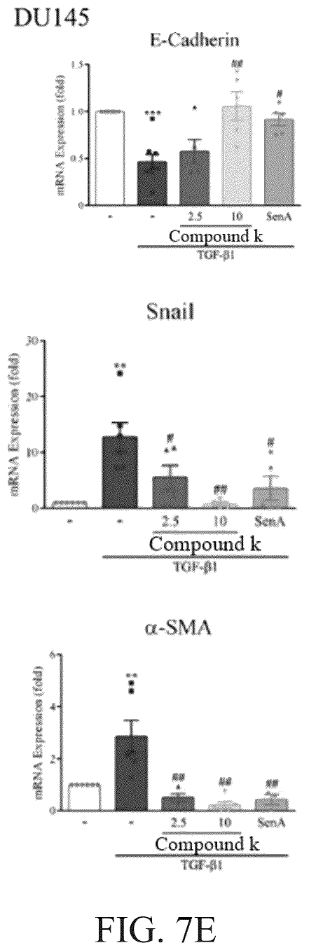

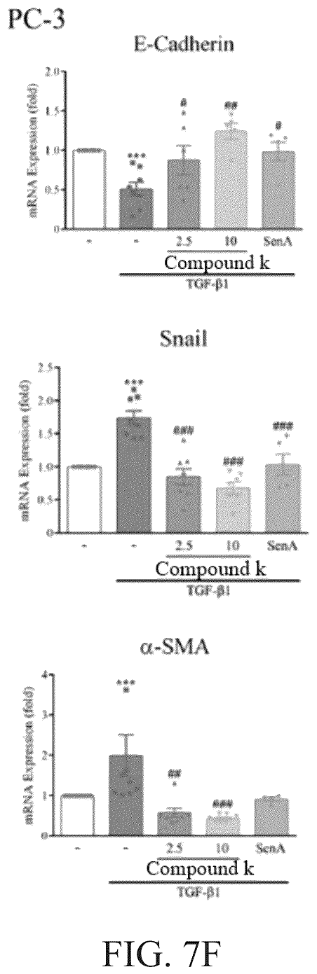

FIGS. 7A-7F shows that compound k significantly inhibited TGF-β1-induced EMT proteins and gene expression.

A previous study indicated that the EMT of pulmonary fibrosis is a process in which alveolar epithelial cells lose contact adhesion, change their shape, transform into myofibroblasts, and exhibit features of invasion, migration, and ECM production (L. Richeldi et al, 2017 Lancet 389 (10082 1941-1952).

TGF-β1 is one of the most potent profibrogenic cytokines. Previous studies identified that TGF-β1 can trigger Smad-dependent signals to increase EMT protein expressions and epithelial migration, ultimately resulting in lung fibrosis (N. G. Frangogiannis et al, 2020 J. Exp. Med. 217 (3), e20190103). The CDK8 inhibitors in this study were investigated to determine how they would modulate the TGF-β1 signaling. As we discovered, TGF-β1 treatment significantly increased myofibroblast markers such as collagen I, α-smooth muscle actin (α-SMA), and Snail expressions in human alveolar epithelial A549 cells; meanwhile, treatment with TGF-β1 also downregulated E-cadherin levels, an epithelial cell marker (FIG. 4A). These results indicated that TGF-β1 treatment triggered EMT progression in our model. Senexin A (a CDK8 inhibitor) and pirfenidone (a clinical drug in idiopathic pulmonary fibrosis treatment) were used for reference compound, both of which displayed slight inhibition of EMT protein levels. Interestingly, significant inhibition of TGF-β1 that triggered EMT protein expressions was observed when A549 epithelial cells were treated with compound k. In contrast, compound b displayed a slight reduction in A549 cells, while compound a displayed no significant reduction in EMT protein levels (FIG. 4A).

Of the compounds tested, compound k displayed the greatest potency than other test compounds and reference agents. This appears consistent with their CDK8 inhibitory activities. Further, the effect on cell viability of A549 cells by compound k was also evaluated. As shown, cell viabilities of treated cells did not significantly change when treated with compound k for 12, 24, and 48 h (FIG. 4B). This was also observed when compound concentrations were increased to 10 μM. This suggests that the inhibition of EMT protein expressions by compound k was not due to cell toxicity.

In addition, cell migration was evaluated when treated with the CDK8 inhibitors. TGF-β1-treated A549 alveolar epithelial cells for 24 h showed a sharper appearance and cell migration; after 48 h of treatment, significant cell migration was observed (FIGS. 5A-5B). Compound k exhibited potent inhibition of cell migration; by contrast, the mild (a) and moderate (b) CDK8 inhibitors and two reference agents displayed less inhibition of cell migration. This suggests that compound k is a more-potent modulator of the EMT in this lung fibrosis model. (FIG. 5A-5B)

Moreover, TGF-β is also a prominent inducer of cancer metastasis. Overexpression of TGF-β is significantly associated with poorer clinical outcome in prostate cancer metastasis (T. Trivedi et al, 2021 Biomolecules 11 (11) 1643). Therefore, functional assays were conducted on prostate cancer cells to explore the potential effects of CDK8 inhibition on tumor cell metastasis. Transitions from clumped, cobblestone-like shapes into scattered and elongated morphological features were observed in DU145 cells in response to TGF-β1 (10 ng/mL) stimulation for 24 h (FIG. 6A). Compound k treatment appeared to reverse these effects. Next, we examined whether compound k inhibited the migration of prostate cancer cells. The results showed significant enhancement of wound closure in response to TGF-β1 treatment for 48 h, and compound k significantly inhibited TGF-β1-induced migration in prostate cancer cells (FIGS. 6B-6C). In addition, transwell assay was conducted to confirm the inhibitory effect of migration by compound. The results showed that compound k treatment significantly inhibited TGF-β1-triggered cell migration in both prostate cancer cells. We further studied the mechanisms underlying the inhibition of cell migration by the compound. As shown, prostate cancer cells that were exposed to TGF-β1 increased several epithelial to mesenchymal transition (EMT) proteins, such as N-cadherin, Snail, and vimentin, and decreased the epithelial marker E-cadherin levels (FIGS. 6D and FIGS. 7A-7D). Compound k treatment attenuated the effects of TGF-β1 by upregulating the epithelial marker E-cadherin and downregulating the mesenchymal markers N-cadherin, Snail, and vimentin (FIGS. 6D and FIGS. 7A-7D). To determine whether such outcomes were the result of transcription inhibition, real-time PCR experiments were also performed. Changes in mRNA levels mirrored those of the proteins (FIG. 7E-7F). A commercial CDK8 inhibitor, senexin A (IC50=280 nM), was used as a reference compound. Compound k exhibited more potent EMT protein inhibitory effects than senexin A (FIGS. 6D and FIGS. 7A-7F). These results indicated that compound k significantly inhibited TGF-β1-induced EMT gene expression and migration in prostate cancer cells.

### Example 7. Compound k Suppressed TGF-β1/Smad Signaling Through Inhibition of RNA Polymerase II and Smad Linker Phosphorylation

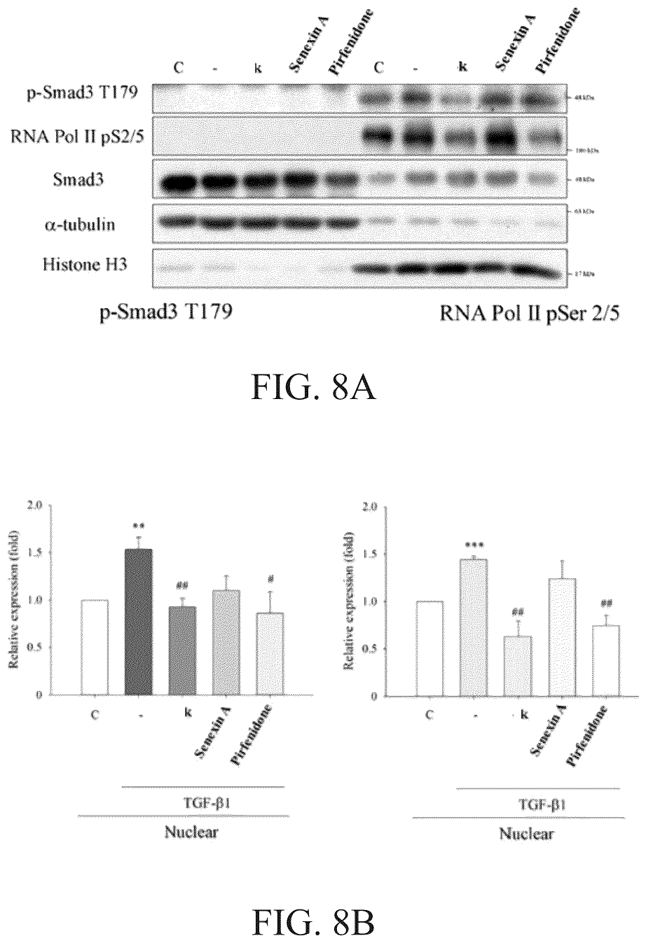

FIGS. 8A-8B show the inhibitory effects of cellular distributions of TGF-β1-induced p-Smad3 T179, RNA polymerase II pS2/S5, and Smad3 after treatment with compound k.

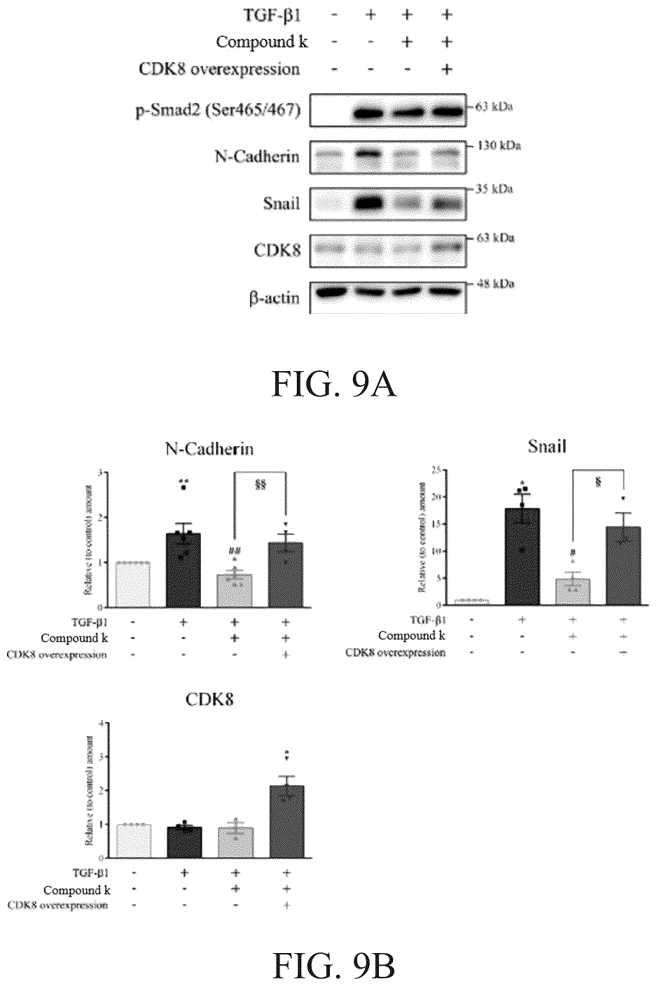

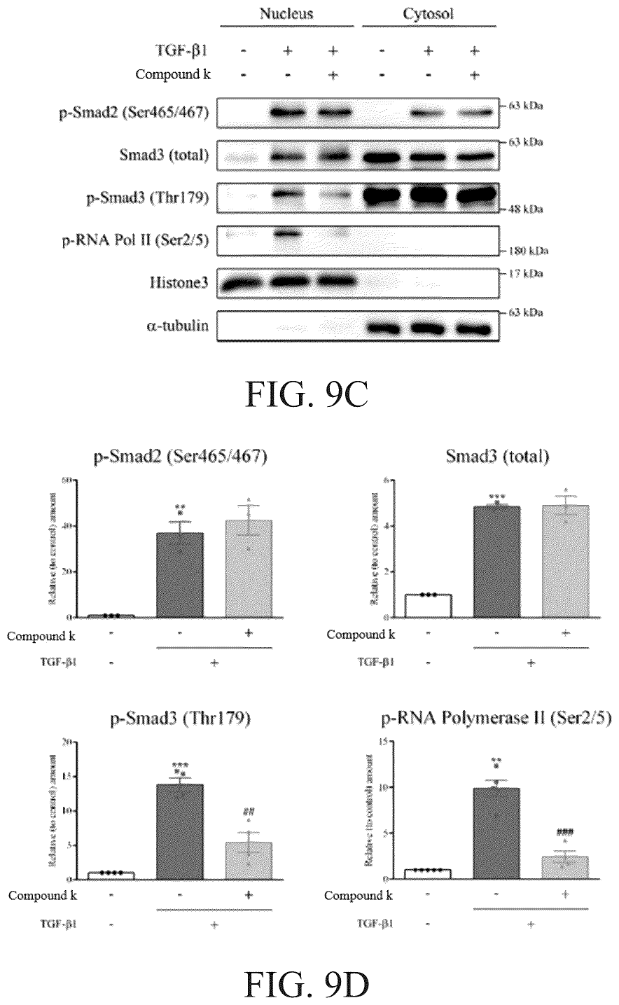

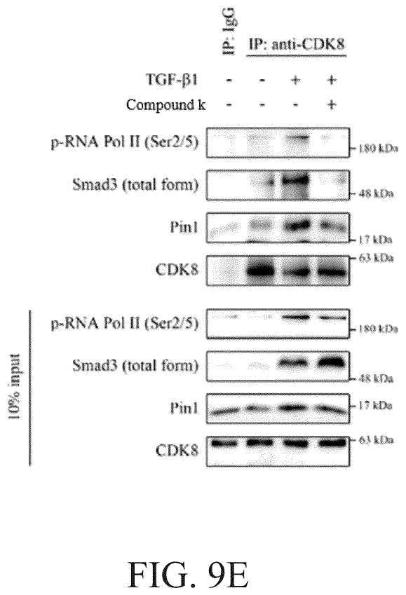

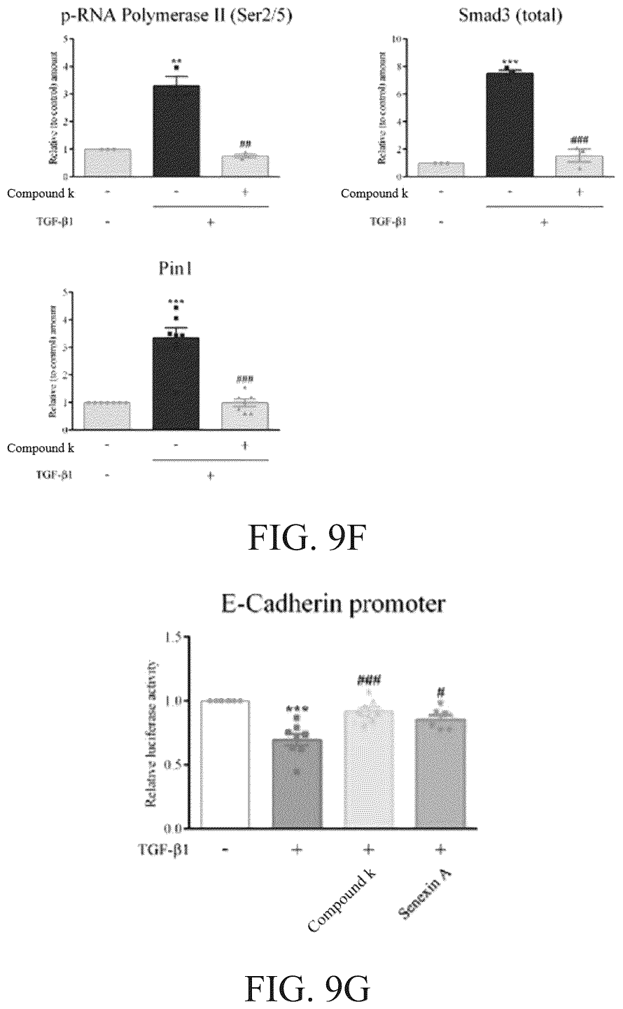

FIGS. 9A-9G show that compound k significantly inhibited TGF-β/Smad/RNA polymerase II signaling.

It is known that TGF-β1 mainly induces Smad pathways to enhance EMT proteins and a redox imbalance, all of which contribute to the pathogenesis of lung fibrosis (Y. Park et al, 2021 Sci. Rep. 11 (1) 4318). Following TGF-β1 binding to its receptor, Smad3 binds with Smad4, is then translocated to the nucleus, and binds to DNA. CDK8 phosphorylates Smad3 at T179, leading to the activation of RNA polymerase II and regulation of ECM protein transcription (J. Massague et al, 2012 Nat. Rev. Mol. Cell Biol. 13 (10) 616-630; E. Aragon et al, 2011 Genes Dev. 25 (12) 1275-1288). The effect of Smad3 phosphorylation and RNA polymerase II activation were assessed when treated with CDK8 inhibitors. After treatment with various compounds, cells were fractionated into cytoplasmic and nuclear fractions, for which α-tubulin and histone H3 were respectively used as markers. As expected, total Smad3 was mainly distributed in the cytoplasmic fraction in a resting condition; only a few Smad3 proteins were observed in the nuclear fraction (FIGS. 8A-8B). Further, a small amount of phosphorylated Smad3 T179 expression was detected in both the cytoplasmic and nuclear fractions. In contrast, TGF-β1 treatment not only significantly increased total Smad3 levels in the nucleus, but also markedly enhanced expressions of p-Smad3 T179 and RNA polymerase II pSer2/5 in the nucleus when compared to the control group. These results indicated that Smad3 proteins were translocated to the nucleus, were phosphorylated at the Thr179 site, and then activated RNA polymerase II through TGF-β treatment in alveolar epithelial A549 cells. Compound k treatment significantly reversed the phosphorylation of p-Smad3 T179 and RNA Pol II pSer2/5; in contrast, two reference agents, senexin A and pirfenidone, produced less-potent inhibition of the phosphorylation of p-Smad3 and RNA Pol II (FIGS. 8A-8B).

Moreover, CDK8 facilitates ligand-induced transcriptional activity through the phosphorylation of transcription factors (I. Menzl et al, 2019 Pharmaceuticals 12 (2) 92). To better understand whether the suppression of migration by the compound involves inhibition of transcriptional regulation by CDK8, we further examined the effects of the compound on the TGF-β/Smad canonical pathway. Upon receptor activation by TGF-β1, the Smad family of signal transducers Smad2 and Smad3 are phosphorylated at the C-terminal domain (e.g., Ser465/467), and these tail-phosphorylated Smads then complex with a comediator Smad4, which translocates to the nucleus. In the nucleus, the CDK8/mediator complex phosphorylates Smad3 at Thr179, which creates high-affinity binding sites for the coactivator Pin1 and other transcriptional partners, followed by active RNA polymerase II S2/S5 to achieve peak transcriptional action and promote EMT and metastasis (C. Alarcon et al, 2009 Cell 139 (4) 757-769). Through interactions with different transcription factors, cofactors, and RNA polymerase II, 2013 Cell 153 (6) 1327-13). As shown, the levels of p-Smad2 (Ser465/467) and the mesenchymal markers N-cadherin and Snail increased in response to 24 h postintervention by TGF-β1; compound k treatment significantly downregulated the expression of mesenchymal markers (FIGS. 9A-9B).

To confirm the participation of CDK8 in this interplay, CDK8 was overexpressed to counteract the inhibitory effects of compound k. Interestingly, the inhibitory effects of the compound were restored in CDK8-overexpressing DU145 cells (FIGS. 9A-9B), indicating the inhibitory effect of the compound on CDK8. Next, the changes in downstream signals following compound treatment were examined. A nuclear extraction protocol was conducted to isolate cytosolic and nuclear fractions, and α-tubulin and histone H3 were used as cytoplasmic and nuclear markers, respectively (FIGS. 9C-9D). TGF-β1 treatment significantly increased p-Smad3 T179 and p-RNA polymerase II S2/5 levels in the nuclear fraction, and compound k markedly suppressed the phosphorylation of Smad3 and RNA polymerase II (FIGS. 9C-9D). We also reconfirmed the interactions between CDK8, transcription factors, RNA polymerase II, and Pin1 coactivator in response to compound k treatment by immunoprecipitation.

TGF-β1 treatment significantly increased the binding of CDK8 to RNA polymerase II (Ser2/5), Smad3, and Pin1, and these interactions were inhibited by compound k treatment (FIGS. 9E-9F). Furthermore, to identify the inhibitory effect of the compound on EMT, we transfected DU145 cells with a CDH1 reporter, which encodes a key epithelial protein marker E-cadherin and evaluated CDH1-driven luciferase expression. As anticipated, TGF-β1 treatment significantly reduced CDH1-driven luciferase expression, and compound k reversed this effect to the control level (FIG. 9G). These results showed that compound k inhibits EMT signals via CDK8 inhibition.

### Example 8. Compounds Reduced TGF-β1-Induced Oxidative Stress

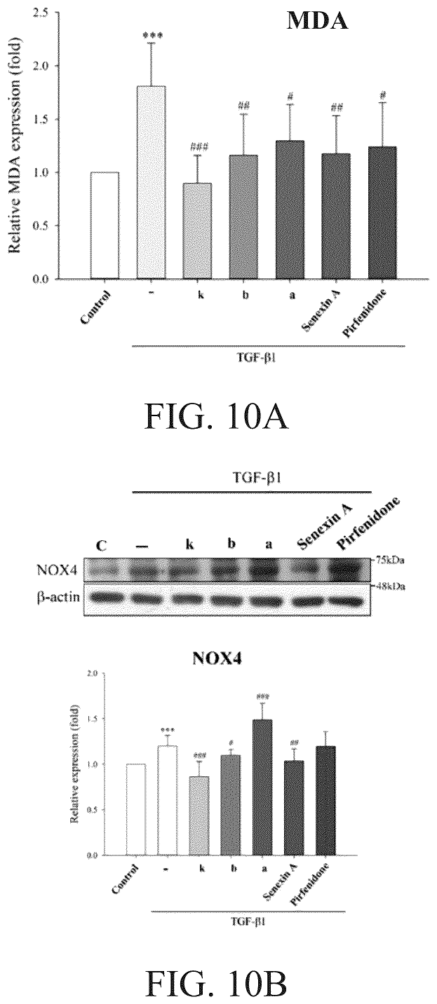

FIGS. 10A-10B show that CDK8 inhibition reduced TGF-β1-induced oxidative stress.

Accumulating evidence also indicated that TGF-β1 increases the production of reactive oxygen species (ROS) by impairing mitochondrial function and inducing NADPH oxidases (NOXs), mainly NOX4, through the Smad pathway that leads to a redox imbalance. A redox imbalance also induces TGF-β1 expression and promotes fibrogenic effects, forming a vicious cycle (R. M. Liu et al, 2015 Redox Biol. (6) 565-577). As shown in FIGS. 10A-10B, TGF-β1 treatment significantly increased oxidative stress, characterized by increased malondialdehyde (MDA. and NOX4 levels in alveolar epithelial A549 cells, and compound k markedly reduced TGF-β1-caused oxidative stress. In contrast, the identified inhibitors, compounds b and a, produced less-potent inhibition of MDA and Nox4 expressions (FIGS. 10A-10B). Together, these results suggested that compound k is a potent CDK8 inhibitor and can potentially modulate signaling pathways associated with fibrogenic responses.

### Example 9. Compound k Hindered TGF-β1-Induced β-Catenin Signaling

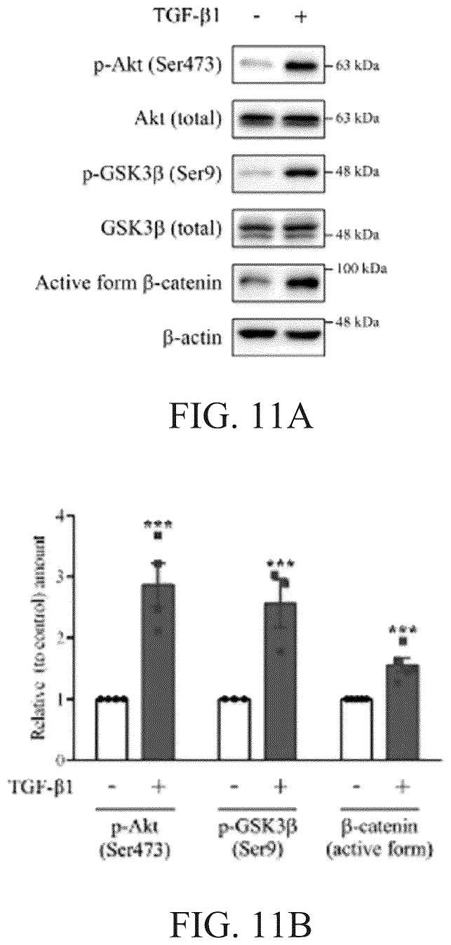

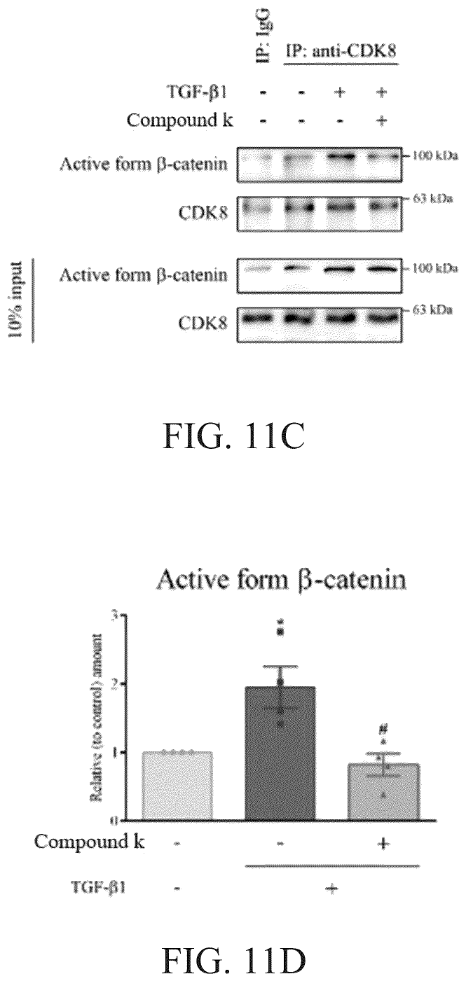

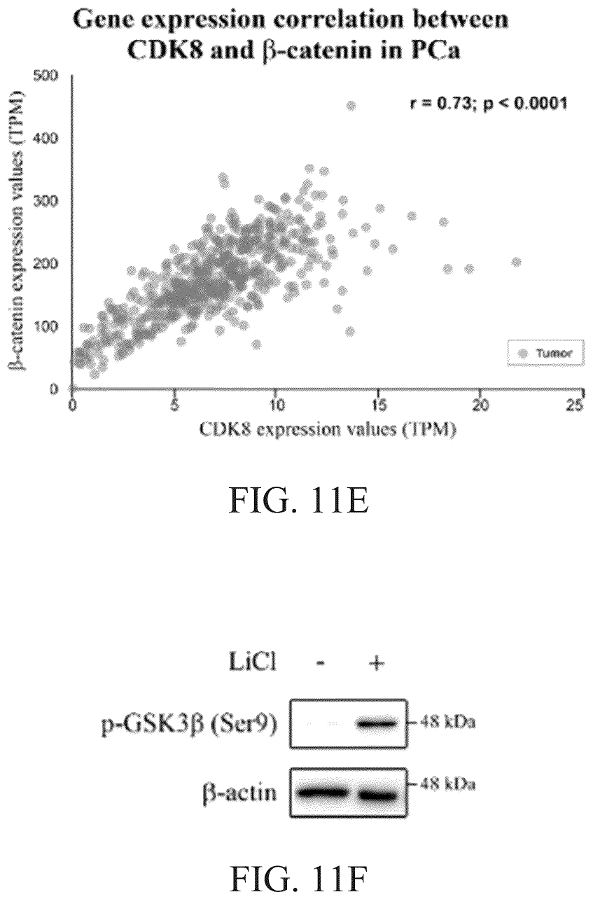

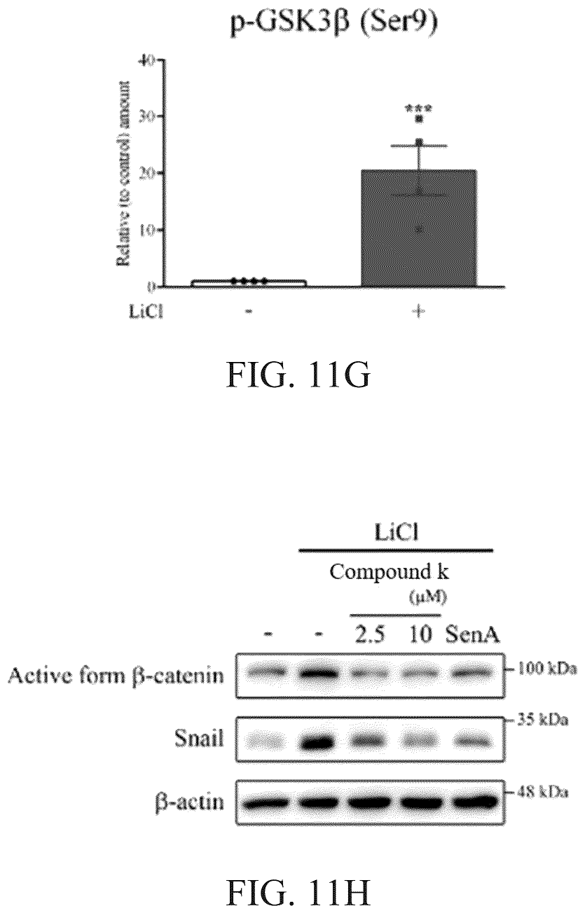

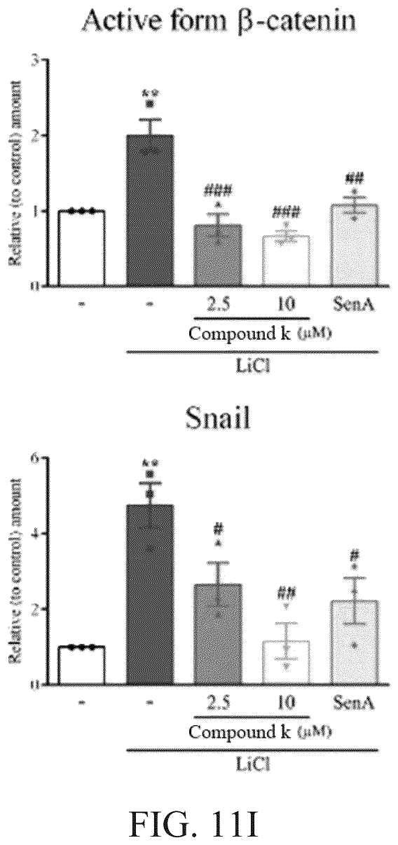

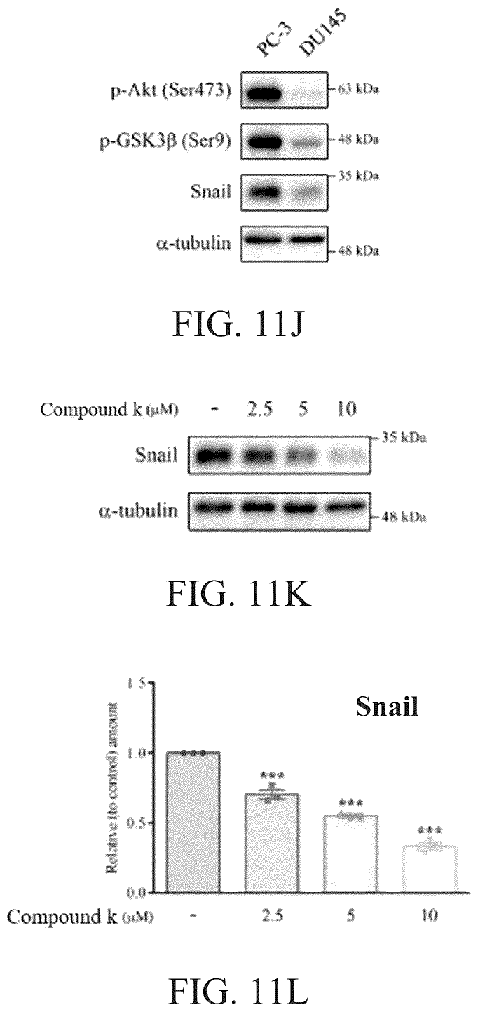

FIGS. 11A-11L show that compound k hindered TGF-β1-induced β-catenin signaling.

In addition, the TGF-β/Akt/glycogen synthase kinase 3B (GSK3β)/β-catenin noncanonical pathway also plays a role in the metastasis of multiple cancers, and a recent study demonstrated that CDK8 kinase activity is required for β-catenin-driven transcription (R. Firestein et al, 2008 Nature 455 (7212) 547-551; A. Hamidi et al, 2017 Sci. Signal. 10 (486), eaal4186). Therefore, we investigated whether β-catenin signaling also plays a role in treatment significantly increased the phosphorylation of Akt at Ser473, GSK38 at Ser9 (the inactive form), and the active form of β-catenin in DU145 prostate cancer cells (FIGS. 11A-11B). The extent of the interaction between the active form of β-catenin and CDK8 increased upon TGF-β treatment in the immunoprecipitation assay and was significantly inhibited by compound treatment (FIGS. 11C-11D), suggesting that CDK8 also plays a role in the regulation of 8-catenin signals. Interestingly, CDK8 and β-catenin gene expression also exhibited a positive correlation in 498 prostate cancer patients in the TCGA database (FIG. 11E). Since LiCl can significantly increase the level of GSK38 phosphorylation at Ser9 (the inactive form) (FIGS. 11F-11G), we inactivated GSK38 with LiCl and evaluated the changes in β-catenin signals and downstream proteins in response to compound treatment. As shown, LiCl-treated DU145 cells showed a significant increase in the levels of the active form of β-catenin and Snail downstream proteins compared to those in control cells; CDK8 inhibition, both with compound k and senexin A, reduced the levels of these factors, and compound k exhibited better efficacy than senexin A (FIGS. 11H-11I). Furthermore, significant p-Akt (Ser473)/p-GSK3β (Ser9) expression was observed in the basal condition of PC-3 cells (FIG. 11J), indicating that activating agent treatment is not required to stimulate this signal in PC-3 cells. Compound k treatment markedly inhibited Snail expression in PC-3 cells (FIGS. 11K-11L). These results support that the compound inhibits 8-catenin signaling and downstream proteins via CDK8 inhibition.

### Example 10. Antimetastatic Properties In Vivo

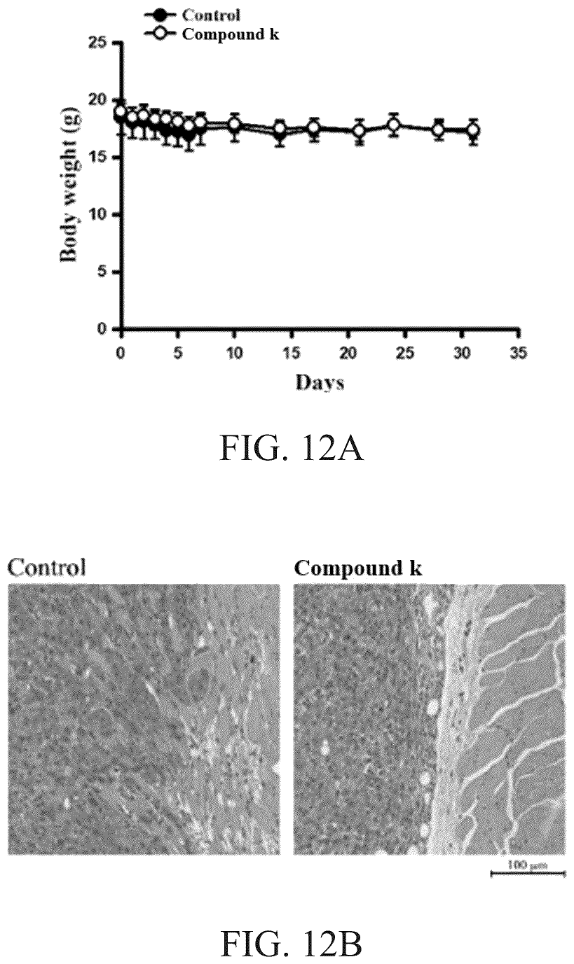

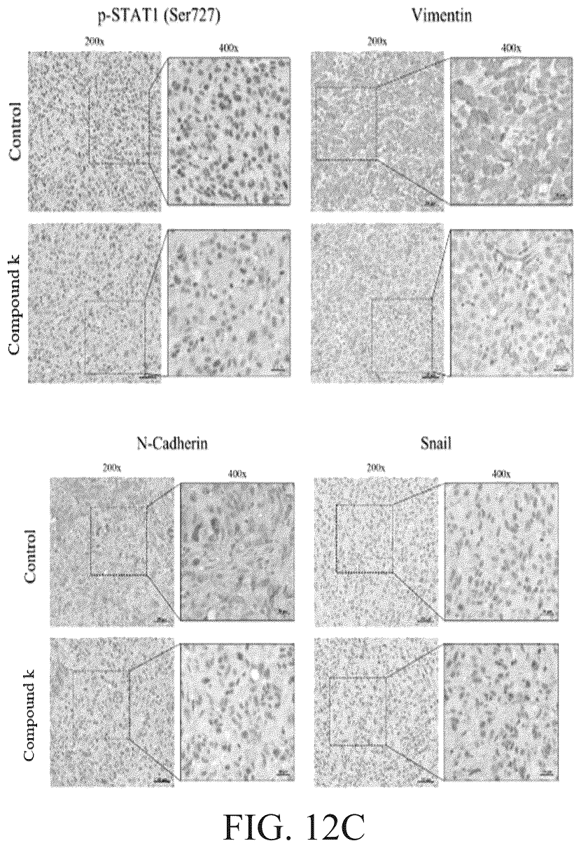

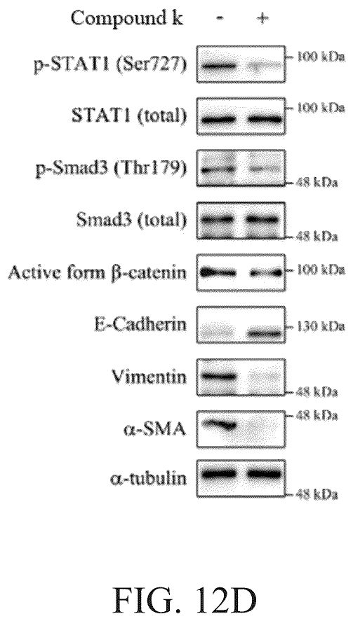

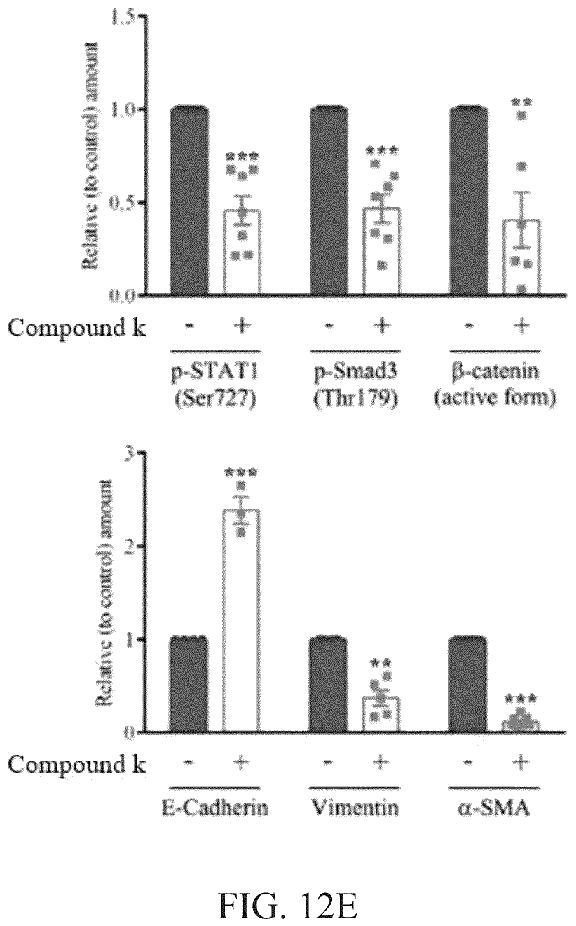

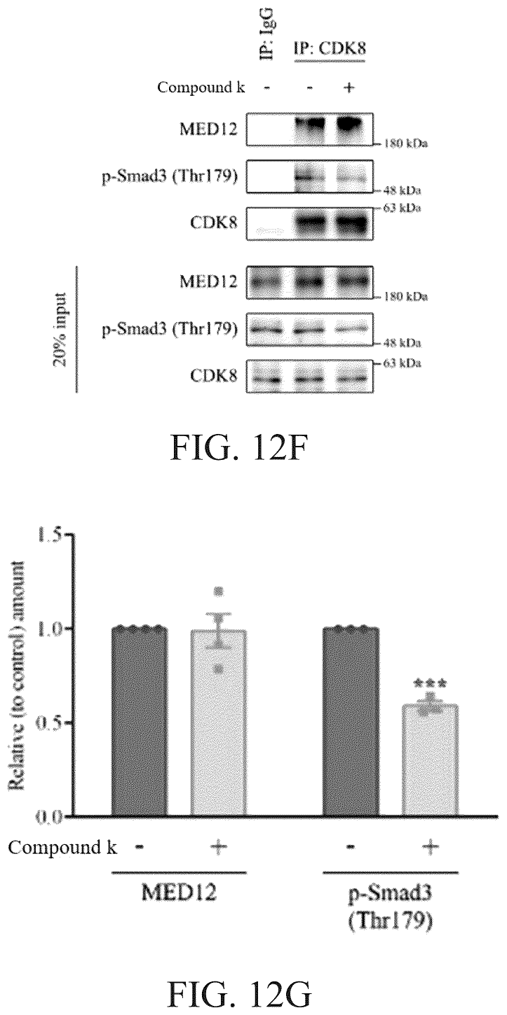

FIGS. 12A-12G show that compound k exhibited anti-invasion effects in vivo.

Compound k was further evaluated for in vivo efficacy in a PC-3 SCID mouse model. The results showed that oral administration of the compound (50 mg/kg, q.d.) induced no apparent body weight changes (FIG. 12A). Furthermore, the margins of the harvested tumors were examined for their invasion into surrounding tissues to identify metastasis. In the H&E staining results, the control group displayed a continuous tumor margin, indicating that tumor cells infiltrated the surrounding tissues (FIG. 12B); however, a distinct boundary was observed between the tumor and normal cells in the compound k-treated group (FIG. 12B). Notably, the integrity of the tumor capsule was preserved in the compound treatment group, and tumor capsule invasion has been linked to worse prognosis and a higher risk of recurrence in clinical studies (A. Buhmeida et al, 2006 Diagn. Pathol. 1:4). According to the IHC staining results, the key EMT proteins vimentin, N-cadherin, and Snail were overexpressed in the control group, whereas the levels of these proteins were significantly decreased in the compound k treatment group (FIG. 12C). In addition, immunoblots of tumor samples showed that compound treatment markedly inhibited the expression of EMT proteins (e.g., vimentin), myofibroblast marker α-SMA, p-Smad3 (Thr179), the active form of β-catenin, CDK8 activity markers (e.g., p-STAT1 Ser727), and increased the expression of epithelial cell markers (e.g., E-cadherin) (FIGS. 12D-12E). Furthermore, an immunoprecipitation assay was conducted to study whether the compound inhibits CDK8 cooperation with the mediator complex to regulate metastatic signals. As shown, significant interactions between CDK8, MED12, and p-Smad3 (Thr179) were observed in the untreated group (FIGS. 12F-12G). Compound k treatment markedly inhibited the binding of CDK8 and p-Smad3 (Thr179) but did not affect the interaction of CDK8 and MED12. Collectively, these results support that compound k inhibits prostate cancer metastasis through CDK8 inhibition.

Given the disclosure above, the compounds described herein may have a great potential in pharmaceutical and medical applications, in particular in the treatment of CDK8-mediated diseases.

It is understood that the foregoing examples are merely illustrative of the present invention. Certain modifications of the articles and/or methods employed may be made and still achieve the objectives of the invention. Such modifications are contemplated as within the scope of the claimed invention.

What is claimed is:

1. A compound of Formula (I): 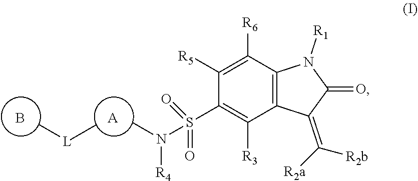
or a pharmaceutically acceptable salt, stereoisomers, enantiomers, prodrugs, hydrates or solvates thereof, wherein:
R¹ is hydrogen, C₁₋₆ alkyl, C₁₋₆ haloalkyl, C₁₋₆ alkoxy, C₁₋₆ haloalkoxy or phenyl;
R^{2a} and R^{2b} each independently are C₁₋₆ alkyl, C₁₋₆ alkenyl or C₁₋₆ alkynyl, which is optionally substituted with halogen, or
R^{2a} and R^{2b} taken together with the carbon atom to which they attach form Ring C, wherein Ring C is 4-6 membered cycloalkyl or 4-6 membered heterocycloalkyl, wherein the 4-6 membered cycloalkyl or 4-6 membered heterocycloalkyl is optionally substituted with phenyl or benzyl, in which said phenyl or benzyl is optionally substituted with halogen or hydroxy;
each of R³, R⁵, and R⁶, independently is, H, C₁₋₆alkyl, C₂₋₆alkenyl, C₂₋₆alkynyl, 6-10 membered aryl, 5-10 membered heteroaryl, 3-8 membered cycloalkyl, 3-8 membered cycloalkenyl, 3-8 membered heterocycloalkyl, 3-8 membered heterocycloalkenyl, halo, cyano, nitro, ORₐ, SRₐ, S(O)Rₐ, SO₂Rₐ, CH═CH—C(O)NR_{b}R_{c}, NHC(O)—CH═CH—C(O)Rₐ, NHC(O)—CH═CH—C(O)NR_{b}R_{c}, SO₂NR_{b}R_{c}, OC(O)Rₐ, C(O)NR_{b}R_{c}, NR_{b}R_{c}, NHC(O)Rₐ, NHC(O)NR_{b}R_{c}, or NHC(S)R_{b}, wherein each of Rₐ, R_{b}, and R_{c}, independently, is H, hydroxy, C₁₋₆alkoxy, 6-10 membered aryloxy, 5-10 membered heteroaryloxy, C₁₋₆alkyl, C₂₋₆alkenyl, C₂₋₆alkynyl, 6-10 membered aryl, 5-10 membered heteroaryl, 3-8 membered cycloalkyl, 3-8 membered cycloalkenyl, 3-8 membered heterocycloalkyl, or 3-8 membered heterocycloalkenyl; and
R⁴ is hydrogen, C₁₋₁₀ aliphatic group, phenyl or 5-6 membered heteroaryl, which is optionally substituted with one or more substituents selected from the group consisting of halogen, hydroxy, nitro, cyano, amino, phenyl, 5-6 membered heteroaryl, C₁₋₆ alkoxy and C₁₋₆ haloalkoxy;
Ring A is phenyl, a diazole, a thiadiazole, a thiazolobenzene or a benzopiperazine, which is optionally substituted with one or more substituents selected from the group consisting of oxo, halogen, C₁₋₆alkyl, phenyl, a diazole and C₁₋₃alkylaminocarbonyl, wherein the substituent C₁₋₆alkyl, phenyl and diazole is optionally further substituted with halogen, C₁₋₃alkyl or any combinations thereof;
-L- is —O—, —NR^{a}—, —C(═O)—, —NR^{a}—C(═O)— or —C(═O)—NR^{a}—, wherein R^{a} has the meaning as defined above; and
Ring B is phenyl, a diazole or pyridine, which is optionally substituted with one or more substituents selected from the group consisting of nitro, halogen, alkyl, pyridine and C₁₋₃alkylaminocarbonyl; or
-L-B is absent.

2. The compound of claim 1, or a pharmaceutically acceptable salt, stereoisomers, enantiomers, prodrugs, hydrates and solvates thereof, wherein R¹ is hydrogen and R^{2a} and R^{2b} each independently are methyl.

3. The compound of claim 1, or a pharmaceutically acceptable salt, stereoisomers, enantiomers, prodrugs, hydrates and solvates thereof, wherein:
Ring A is phenyl, a diazole, a thiadiazole, a thiazolobenzene or a benzopiperazine, which is optionally substituted with one or more substituents selected from the group consisting of oxo, halogen, C₁₋₆alkyl, phenyl, a diazole and C₁₋₃alkylaminocarbonyl, wherein the substituent C₁₋₆alkyl, phenyl and diazole is optionally further substituted with halogen, C₁₋₃alkyl or any combinations thereof;
-L- is —O—, —NR^{a}— or —C(═O)—; and
Ring B is phenyl, a diazole or pyridine, which is optionally substituted with one or more substituents selected from the group consisting of halogen, alkyl and C₁₋₃alkylaminocarbonyl; or
-L-B is absent.

4. The compound of claim 1, or a pharmaceutically acceptable salt, stereoisomers, enantiomers, prodrugs, hydrates and solvates thereof, wherein Ring A is phenyl optionally substituted with one or more substituents selected from the group consisting of halogen and C₁-6alkyl; L is —O—, —NH—, —C(═O)— or —C(═O)—NR^{a}— wherein Ring A is linked to the N atom; and Ring B11 is phenyl or pyridine which is optionally substituted with one or more substituents selected from the group consisting of nitro, halogen, C₁₋₆alkyl and C₁₋₃alkylaminocarbonyl.

5. The compound of claim 1, or a pharmaceutically acceptable salt, stereoisomers, enantiomers, prodrugs, hydrates and solvates thereof, wherein Ring A is phenyl, a diazole or a thiadiazole, which is substituted with one or more substituents selected from the group consisting of phenyl, a diazole or C₁₋₃alkylaminocarbonyl, and the substituent phenyl and diazole is optionally further substituted with C₁₋₃alkyl; and -L-B is absent.

6. The compound of claim 1, or a pharmaceutically acceptable salt, stereoisomers, enantiomers, prodrugs, hydrates and solvates thereof, wherein Ring A is a thiazolobenzene or a benzopiperazine, which is optionally substituted with one or more substituents selected from the group consisting of oxo, halogen and alkyl; and -L-B is absent.

7. The compound of claim 1, or a pharmaceutically acceptable salt, stereoisomers, enantiomers, prodrugs, hydrates and solvates thereof, wherein Ring A is phenyl, which is optionally substituted with one or more substituents selected from the group consisting of halogen and alkyl; -L-is-NH—; and Ring B is pyridine which is optionally substituted with one or more substituents selected from the group consisting of halogen and C₁₋₆alkyl.

8. The compound of claim 1, or a pharmaceutically acceptable salt, stereoisomers, enantiomers, prodrugs, hydrates and solvates thereof, wherein Ring A is substituted with halogen.

9. The compound of claim 1, or a pharmaceutically acceptable salt, stereoisomers, enantiomers, prodrugs, hydrates and solvates thereof, wherein Ring A is phenyl substituted with halogen; -L-is-O—; Ring B is pyridine substituted with C₁₋₃alkylaminocarbonyl.

10. The compound of claim 1, or a pharmaceutically acceptable salt, stereoisomers, enantiomers, prodrugs, hydrates and solvates thereof, wherein the compound has formula (II): 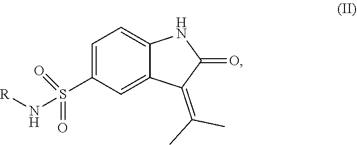
wherein R is selected from the group consisting of:

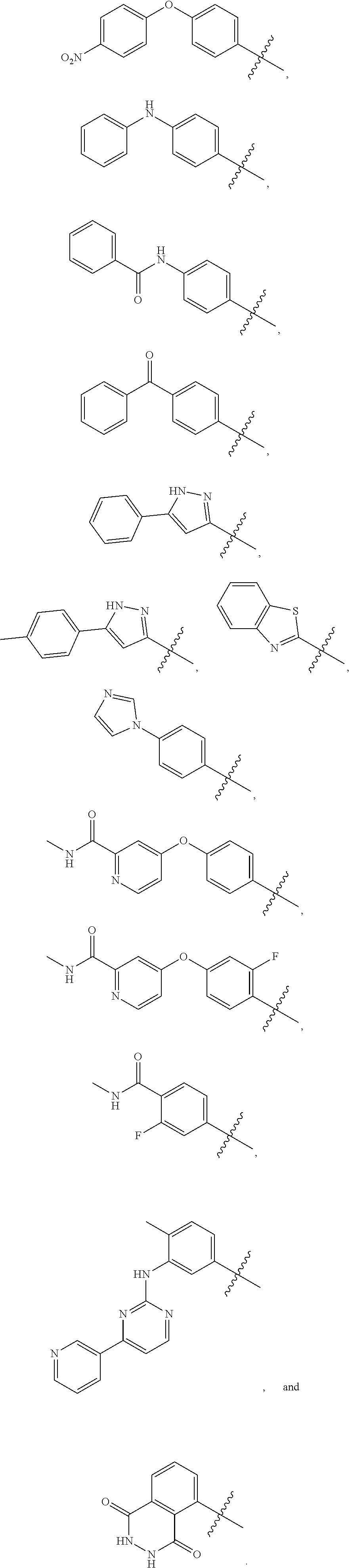

11. The compound of claim 1, or a pharmaceutically acceptable salt, stereoisomers, enantiomers, prodrugs, hydrates and solvates thereof, wherein the compound is selected from the group consisting of:
N-(4-(4-Nitrophenoxy)phenyl)-2-oxo-3-(propan-2-ylidene)indoline-5-sulfonamide,
2-oxo-N-(4-(phenylamino)phenyl)-3-(propan-2-ylidene)indoline-5-sulfonamide,
N-(4-((2-oxo-3-(propan-2-ylidene)indoline)-5-sulfonamido)phenyl)benzamide,
N-(4-benzoylphenyl)-2-oxo-3-(propan-2-ylidene)indoline-5-sulfonamide,
2-oxo-N-(5-phenyl-1H-pyrazol-3-yl)-3-(propan-2-ylidene)indoline-5-sulfonamide,
2-oxo-3-(propan-2-ylidene)-N-(5-(p-tolyl)-1H-pyrazol-3-yl)indoline-5-sulfonamide,
2-oxo-N-(5-phenyl-1,3,4-thiadiazol-2-yl)-3-(propan-2-ylidene)indoline-5-sulfonamide,
N-(benzo[d]thiazol-2-yl)-2-oxo-3-(propan-2-ylidene)indoline-5-sulfonamide,
N-(4-(1H-imidazol-1-yl)phenyl)-2-oxo-3-(propan-2-ylidene)indoline-5-sulfonamide,
N-methyl-4-(4-((2-oxo-3-(propan-2-ylidene)indoline)-5-sulfonamido)phenoxy)picolinamide,
4-(3-fluoro-4-((2-oxo-3-(propan-2-ylidene)indoline)-5-sulfonamido)phenoxy)-N-methylpicolinamide,
2-fluoro-N-methyl-4-((2-oxo-3-(propan-2-ylidene)indoline)-5-sulfonamido)benzamide,
N-(4-methyl-3-((4-(pyridin-3-yl)pyrimidin-2-yl)amino)phenyl)-2-oxo-3-(propan-2-ylidene)indoline-5-sulfonamide, and
N-(1,4-dioxo-1,2,3,4-tetrahydrophthalazin-5-yl)-2-oxo-3-(propan-2-ylidene)indoline-5-sulfonamide.

12. A pharmaceutical composition comprising a compound of claim 1 or a pharmaceutically acceptable salt, stereoisomers, enantiomers, prodrugs, hydrates and solvates thereof, and a pharmaceutically acceptable carrier.

13. The pharmaceutical composition of claim 12, further comprising an additional therapeutic agent.

14. The pharmaceutical composition of claim 13, wherein the additional therapeutic agent is selected from the group consisting of: an anti-fibrosis agent, an alkylating agent, an antibiotic; an antimetabolite; an antibody therapy agent; a hormone or hormone antagonist; a taxane; a retinoid; an alkaloid; an antiangiogenic agent; a topoisomerase inhibitor; a kinase inhibitor; a targeted signal transduction; a biological response modifier; a chemotherapeutic agent; an Hsp90 inhibitor; a farnesyltransferase inhibitors; an aromatase inhibitor; anindoleamine 2,3-dioxygenase (IDO) inhibitor; a histone acetyltransferase (HAT) inhibitor; a histone deacetylase (HDAC) inhibitor; a sirtuin (SIRT) inhibitor; a Bromodomain and Extra-Terminal motif (BET) inhibitor; an anticancer agent; and any combinations thereof.

15. A method of inhibiting CDK8 in a subject, comprising administering a therapeutically effective amount of the compound of claim 1 or a pharmaceutically acceptable salt, stereoisomers, enantiomers, prodrugs, hydrates and solvates thereof to the subject.

16. A method of preventing or treating an inflammatory condition or fibrosis diseases, comprising administering a therapeutically effective amount of the compound of claim 1 or a pharmaceutically acceptable salt, stereoisomers, enantiomers, prodrugs, hydrates and solvates thereof to a subject in need thereof.

17. The method of claim 16, wherein the an inflammatory condition or fibrosis diseases are selected from the group consisting of Type 1 diabetes graft-versus-host disease, inflammatory bowel disease, psoriasis, psoriatic arthritis, Hashimoto's thyroiditis, food allergy, HCV vasculitis, alopecia areata, systemic lupus erythematosus, multiple sclerosis, rheumatoid arthritis, skin fibrosis, lung fibrosis, renal fibrosis, liver fibrosis, intestinal fibrosis, cystic fibrosis, carciac fibrosis, uterine leiomyoma, adenomyosis and any combinations thereof.

18. The method of claim 17, wherein the lung fibrosis is idiopathic pulmonary fibrosis.

19. A method of preventing or treating tumor or cell proliferative disease, comprising administering a therapeutically effective amount of the compound of claim 1 or a pharmaceutically acceptable salt, stereoisomers, enantiomers, prodrugs, hydrates and solvates thereof to a subject in need thereof.

20. The method of claim 19, wherein the tumor or cell proliferative disease is selected from the group consisting of brain cancer, lung cancer, colon cancer, epidermoid cancel, squamous cell cancer, bladder cancer, gastric cancer, pancreatic cancer, breast cancer, head cancer, neck cancer, renal cancer, kidney cancer, liver cancer, ovarian cancer, prostate cancer, colorectal cancer, uterine cancer, rectal cancer, oesophageal cancer, testicular cancer, thyroid cancer, melanoma, uveal melanoma, acute myelogenous, leukemia, acute myeloid leukemia, multiple myeloma, chronic myelogneous leukemia, myeloid cell leukemia, glioma, Kaposi's saRboma, human laryngeal squamous cell caRbinoma and any combinations thereof.

21. The method of claim 20, wherein the tumor or cell proliferative disease is a prostate cancer.
